SEQUENCE LISTING (24 sequences)

SEQ ID NO: 1 - protein, 130 aa, Homo sapiens
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIIS

SEQ ID NO: 2 - protein, 130 aa, synthetic construct
APTSSSTKKTQLQLEHLLLDLQMILNGINNYKNPKLHLMLSYGFYMPKKATELKHLQCLEEELKPLEEVLNLAQSKNFHLRPRDLISNINVIVLELKGSETTFMCEYADETATIVEFLNRWITFCQSIIS

SEQ ID NO: 3 - protein, 148 aa, Mus musculus
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSP

SEQ ID NO: 4 - protein, 135 aa, Rattus norvegicus
APTSSPAKETQQHLEQLLLDLQVLLRGIDNYKNLKLPMMLTFKFYLPKQATELKHLQCLENELGALQRVLDLTQSKSFHLEDAGNFISNIRVTVVKLKGSENKFECQFDDEPATVVEFLRRWIAICQSIISTMTQ

SEQ ID NO: 5 - protein, 134 aa, Sus scrofa
APTSSSTKNTKKQLEPLLLDLQLLLKEVKNYENADLSRMLTFKFYMPKQATELKHLQCLVEELKALEGVLNLGQSKNSDSANIKESMNNINVTVLELKGSETSFKCEYDDETVTAVEFLNKWITFCQSIYSTLT

SEQ ID NO: 6 - protein, 135 aa, Vulpes vulpes
APITSSSTKETEQQMEQLLLDLQLLLNGVNNYENPQLSRMLTFKFYTPKKATEFTHLQCLAEELKNLEEVLGLPQSKNVHLTDTKELISNMNVTLLKLKGSETSYNCEYDDETATITEFLNKWITFCQSIFSTLT

SEQ ID NO: 7 - protein, 135 aa, Canis lupus; identical to SEQ ID NO: 6

SEQ ID NO: 8 - protein, 134 aa, Macaca mulatta
APTSSSTKKTQLQLEHLLLDLQMILNGINNYKNPKLTRMLTFKFYMPKKATELKHLQCLEEELKPLEEVLNLAQSKNFHLRDTKDLISNINVIVLELKGSETTLMCEYADETATIVEFLNRWITFCQSIISTLT

SEQ ID NO: 9 - protein, 130 aa, synthetic construct
APTSSSTKKTQLQLEHLLLDLQMILNGINNYKNPKLSAMLDFGFYMPKKATELKHLQCLEEELEPLEEVLNLAQSKNFHLRPRDLISNINVIVLELKGSETTFMCEYADETATIVEFLNRWITFCQSIIS

SEQ ID NO: 10 - protein, 130 aa, synthetic construct
APTSSSTKKTQLQLEHLLLDLQMILNGINNYKNPKLSLMLGYGFYMPKKATELKHLQCLEEELKPLEEVLNLAQSKNFHLRPRDLISNINVIVLELKGSETTFMCEYADETATIVEFLNRWITFCQSIIS

SEQ ID NO: 11 - protein, 130 aa, synthetic construct
APTSSSTKKTQLQLEHLLLDLQMILNGINNYKNPKLSVMLGFGFYMPKKATELKHLQCLEEELKPLEEVLNLAQSKNFHLRPRDLISNINVIVLELKGSETTFMCEYADETATIVEFLNRWITFCQSIIS

SEQ ID NO: 12 - protein, 130 aa, synthetic construct
APTSSSTKKTQLQLEHLLLDLQMILNGINNYKNPKLRVMLGFGFYMPKKATELKHLQCLEEELKPLEEVLNLAQSKNFHLRPRDLISNINVIVLELKGSETTFMCEYADETATIVEFLNRWITFCQSIIS

SEQ ID NO: 13 - protein, 130 aa, synthetic construct
APTSSSTKKTQLQLEHLLLDLQMILNGINNYKNPKLSIMLGFGFYMPKKATELKHLQCLEEELKPLEEVLNLAQSKNFHLRPRDLISNINVIVLELKGSETTFMCEYADETATIVEFLNRWITFCQSIIS

SEQ ID NO: 14 - DNA, 21 bp, synthetic construct
  source 1..21: mol_type = other DNA
agcggtgggggcggttctctg

SEQ ID NO: 15 - DNA, 21 bp, synthetic construct
  source 1..21: mol_type = other DNA
tcgagcaagtcttcttcggag

SEQ ID NO: 16 - DNA, 80 bp, synthetic construct
  source 1..80: mol_type = other DNA
agcggtgggggcggttctctggaagttctgttccagggtccgagcggcggatccgcacctacttcaagttctacaaagaa

SEQ ID NO: 17 - DNA, 87 bp, synthetic construct
  source 1..87: mol_type = other DNA
gcacctacttcaagttctacaaagaaaacacagctacaactggagcatttactgctggatttacagatgattttgaatggaattaat

SEQ ID NO: 18 - DNA, 100 bp, synthetic construct
  source 1..100: mol_type = other DNA
  misc_feature 38..46
  misc_feature 53..58
gatgtttcagttctgtggccttcttgggcatgtaaaaahnahnahngagcatahnahngagtttgggattcttgtaattattaattccattcaaaatcat

SEQ ID NO: 19 - DNA, 100 bp, synthetic construct
  source 1..100: mol_type = other DNA
  misc_feature 30..38
  misc_feature 48..50
  misc_feature 54..59
ccacagaactgaaacatcttcagtgtctandtndtndtctcaaacctndtgagndtndtctaaatttagctcaaagcaaaaactttcacttaagacccag

SEQ ID NO: 20 - DNA, 85 bp, synthetic construct
  source 1..85: mol_type = other DNA
catgaatgttgtttcagatccctttagttccagaactattacgttgatattgctgattaagtccctgggtcttaagtgaaagttt

SEQ ID NO: 21 - DNA, 68 bp, synthetic construct
  source 1..68: mol_type = other DNA
  misc_feature 35..40
gggatctgaaacaacattcatgtgtgaatatgctndtndtacagcaaccattgtagaatttctgaaca

SEQ ID NO: 22 - DNA, 62 bp, synthetic construct
  source 1..62: mol_type = other DNA
agtcagtgttgagatgatgctttgacaaaaggtaatccatctgttcagaaattctacaatgg

SEQ ID NO: 23 - DNA, 74 bp, synthetic construct
  source 1..74: mol_type = other DNA
tcgagcaagtcttcttcggagataagcttttgttcgccaccagaagcggccgcagtcagtgttgagatgatgct

SEQ ID NO: 24 - protein, 133 aa, Homo sapiens [sequence omitted]

(19) United States
(12) Patent Application Publication
(10) Pub. No.: US 2024/0352084 A1
(43) Pub. Date: Oct. 24, 2024

(54) THERAPEUTIC MUTEINS

(71) Applicants: University of Dundee, Dundee (GB); Centre Hospitalier Régional Universitaire de Lille, Lille (FR); The Université de Lille, Lille (FR); Institut National de la Santé et de al Recherche Médicale (INSERM), Paris (FR)

(72) Inventors: Ignacio Moraga GONZALEZ, Dundee (GB); Suman MITRA, Lille Cedex (FR); Silvia GAGGERO, Lille (FR)

(21) Appl. No.: 18/290,928

(22) PCT Filed: Jul. 21, 2022
(86) PCT No.: PCT/EP2022/070548

§ 371 Date: Jan. 22, 2024

(30) Foreign Application Priority Data
Jul. 22, 2021 (GB) 2110547.3
Apr. 19, 2022 (FR) FR2203600

(51) Int. Cl. C07K 14/55; A61K 38/00; A61P 35/00

(52) U.S. Cl. CPC C07K 14/55; A61P 35/00; A61K 38/00; C07K 2319/30

(57) ABSTRACT

Disclosed are modified cytokines which, relative to wild-type forms, comprise one or more amino acid modifications. Relative to the activity of wild type cytokines, these modified cytokines exhibit enhanced activity at an acidic pH and often reduced activity at neutral pH. The disclosed modified cytokines are for use in medicine and/or for the treatment and/or prevention of an immunological condition or cancer.

### FIELD

The present invention provides modified molecules with improved activity at acidic pH for use in the treatment of a range of diseases and/or conditions. In particular, the disclosure provides modified cytokines, including, IL-2, for use in treating cancer.

### BACKGROUND

The tumour microenvironment (TME) critically contributes to tumour differentiation and immune evasion, thus counteracting cytokine-induced anti-tumour responses¹. The cellular and molecular bases that define the TME immune-suppressive properties have been extensively studied^{2,3}. However, how the unique physico-chemical properties affect cytokine responses remains very poorly explored. A hallmark of the TME is acidosis. Overproduction of lactic acid by the tumour cells, due to their high glycolytic activity, results in an acidic environment with pH values around 6.2-6.5, contrasting with pH 7.4 found in normal tissues¹⁴. How the acidic TME influences cytokine-receptor binding and cytokine signalling is not known at the moment.

The interleukin-2 (IL-2) cytokine serves as a powerful master regulator of immune activity, making IL-2 a powerful medium to manipulate the immune response to better fight diseases. On resting lymphocytes, IL-2 signals via intermediate affinity IL-2 receptors (Kd ˜10-9

M) consisting of IL-2RR and IL-2Ry, whereas activated lymphocytes additionally express IL 2Ra, which combines with IL-2RP and IL-2Ry to form high affinity receptors (Kd ˜10-11

M), and respond strongly to IL-2 in vivo and mount effective tumour regression 5. Accordingly, IL-2 has been used in the clinic as part of immunotherapies for malignancies for three decades 6. However, partial efficacy and high toxicity has hindered its wider use 6, mostly as a consequence of the broad pleiotropism of IL-2 including its role in simultaneous promotion of both effector and regulatory T (Treg) cells. Accordingly, numerous efforts are ongoing to manipulate IL-2 activity to selectively favour the growth of the effector cells for the treatment of cancer. Yet how the extracellular chemical environment (such as acidic pH that is found in intra-tumoral space) influence IL-2 activity is poorly understood.

Early studies, where acidic pHs were used to disrupt IL-2 binding from the surface of T lymphocytes, suggested that this cytokine is sensitive to pH changes7. However, which of the IL-2 bound receptors is pH sensitive and how acidic pHs affect IL-2 responses is not known.

### SUMMARY

The present disclosure provides modified cytokines which, relative to wild-type forms, comprise one or more amino acid modifications (for example one or more amino acid substitutions). The inventors have discovered that relative to the activity of wild type cytokines, these modified cytokines exhibit enhanced activity at an acidic pH and often reduced activity at neutral pH. Modified cytokines are also referred to as cytokine muteins in the present disclosure.

The tumour microenvironment (TME) critically contributes to tumour differentiation and immune evasion and can counteract certain cytokine-induced anti-tumour responses. A hallmark of the TME is acidosis. Overproduction of lactic acid by the tumour cells, due to their high glycolytic activity, results in an acidic environment with pH values around 6.2-6.5; this contrasts with the neutral pH (e.g. pH 7.4) found in normal tissues.

The activity of the certain cytokines, for example, interleukin 2 (IL-2), is critical for the development and maintenance of aspects of the host immune response to disease, including, for example, T cell immunity. Cytokines can drive the expansion and induction of immune cells and processes. However, cytokine function may be sensitive to changes in pH. For example, the binding between a cytokine and its receptor may be a pH sensitive or dependent process. As noted above, a characteristic of certain diseases, including cancer, is the generation of an acidic microenvironment which can adversely influence cytokine receptor binding and may ultimately reduce the efficacy of any cytokine-based therapeutic.

As noted, a cytokine which is sensitive to pH (i.e. a cytokine which exhibits reduced activity/receptor binding under acidic conditions) may be modified by alteration (e.g. substitution) of one or more of the amino acids of the wild-type primary sequence. Modified cytokines according to this disclosure may exhibit enhanced activity at an acidic pH and/or reduced activity at a neutral pH. This feature makes the modified cytokines useful in medicine and in particular in the treatment and/or prevention of immunological diseases and/or cancer.

A modified cytokine with therapeutic potential—e.g. for use in medicine, may be identified or obtainable by a method comprising:
modifying a wild type cytokine sequence to generate a modified cytokine; contacting the modified cytokine with a ligand or cell; and
determining whether or not the modified cytokine binds the ligand and/or activates the cell, wherein a modified cytokine which binds the ligand and/or activates the cell may be used in medicine or for the treatment and/or prevention of an immunological condition or cancer.

The step of contacting the modified cytokine with a ligand may comprise contacting the modified cytokine with a ligand fragment, wherein the ligand fragment is a cytokine binding fragment. Similarly, the cell may express the ligand and/or a cytokine binding fragment thereof.

A method of identifying a cytokine mutein, preferably a pH-resistant cytokine mutein, may further comprise a step of generating a library comprising nucleic acids encoding cytokine muteins or fragments thereof, wherein the cytokine muteins comprise one or more amino acid substitutions (including, for example, conservative substitutions); (ii) one or more amino acid deletions; (iii) one or more amino acid additions; and (iv) one or more sequence inversions (all of which are described/defined later in this specification).

The mutations may be made within at least one residue which is involved in the binding of the cytokine to its corresponding ligand(s) or receptor(s). Residues involved in the binding profiles of the different cytokines can be determined by the analysis of structural of functional interaction data for those cytokines and their receptors. Mutations may be random mutations or predefined mutations.

The method may further comprise a step of expressing the nucleic acid library to obtain a cytokine mutein library. The cytokine mutein(s) comprised in the library may be expressed on the surface of an expression vehicle such as a cell, a virus of a phage, for example a yeast cell.

A cytokine for modification (by any of the methods or procedures explained herein) may be selected from granulocyte-macrophage colony-stimulating factor (GM-CSF), macrophage colony-stimulating factor (M-CSF), IL-6, IL-11, IL-12, growth hormone (GF1), erythropoietin (EPO), prolactin (PRL), leukemia inhibitory factor (LIF), oncostatin (OSM), thrombopoietin (TPO) or a functional fragment/variant of any of these cytokines.

In one teaching, the cytokine for modification may be CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CCL1e, CCL2, CCL3, CCL3L1, CCL4, CCL5, CCL6, CCL7, CCL8, CCL9/10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CX3CL1, XCL1, XCL2 or a functional fragment/variant of any of these cytokines.

The cytokine for modification may be selected from the group consisting of IFN-a (alpha), IFN-b (beta), IFN-g (gamma), IFN-e (epsilon), IFN-k (kappa), IFN-w (omega), IFN-t (tau), IFN-z (zeta), IFN-d (delta), IFN-1 (lambda) or a functional fragment/variant of any of these cytokines.

The cytokine for modification may comprise a functional fragment or variant of IFN-a (alpha), IFN-b (beta), IFN-g (gamma), IFN-e (epsilon), IFN-k (kappa), IFN-\acute{ω} (omega), IFN-t (tau), IFN-z (zeta), IFN-d (delta), or IFN-1 (lambda).

In another teaching the cytokine for modification may be IL-1, IL-1α, IL-1β, IL-1ra, IL-2, IL-3, IL-4, IL-5, IL-6, IL-7, IL-8, IL-9, IL-10, IL-11, IL-12, IL-13, IL-14, IL-15, IL-16, IL-17A, IL-17B, IL-17C, IL-17D, IL-17E, IL-17F, IL-17L, IL-17A/L, IL-18, IL-19, IL-20, IL-21, IL-22, IL-23, IL-24, IL-25, IL-26, IL-27, IL-28A, IL-28B, IL-29, IL-30, IL-31, IL-32, IL-33, IL-34, IL-35, IL-36, IL-37 or a functional fragment/variant of any of these cytokines.

The cytokine for modification may be granulocyte-macrophage colony-stimulating factor (GM-CSL), macrophage colony-stimulating factor (M-CSL), tumor necrosis factor alpha (TNL-a), transforming growth factor beta (TGL-b), ILN-g (gamma), IL-2, IL-3, IL-4, IL-5, IL-6, IL-7, IL-8, IL-12 or a functional fragment/variant of any of these cytokines.

In one teaching the cytokine may be selected from the group consisting of TNF-a (alpha), TNF-b (beta), TNF-g (gamma), CD252, CD154, CD178, CD70, CD153, 4-1BB-L, LTa, iTβ, LIGHT, TWEAK, APRIL, BAFF, TL1A, GITRL, OX40L, CD40L, FASL, CD27L, CD30L, 4-1BBL, TRAIL, FLT3 ligand, G-CSF, GM-CSF, IFNa/β/ω, IFNy, LIF, M-CSF, MIF, OSM, Stem Cell Factor, TGFpi, TGFp2, TGF33, TSLP ligand, TRAIL, RANKL, APO3L, CD256, CD257, CD258, TL1, AITRL, EDA1 or a functional fragment/variant of any of these cytokines. The cytokine may be TNF-a (alpha), TNF-b (beta), TNF-g (gamma), CD252, CD154, CD178, CD70, CD153, 4-1BB-L, TRAIL, RANKL, AP03L, CD256, CD257, CD258, TL1, AITRL, EDAL or a functional fragment/variant of any of these cytokines. In a preferred embodiment the cytokine is an interleukin, more preferably IL-2 or IL-10.

The step of modifying a wild type cytokine may comprise:
(i) substituting an amino acid of the wild-type primary sequence with another amino acid. A substitution of this type may be a conservative substitution; and/or
(ii) deleting an amino acid from the wild-type primary sequence with another amino acid; and/or
(iii) adding an amino acid to the wild-type primary sequence; and/or
(iv) inverting part of the wild-type primary amino acid sequence.

The step of contacting the modified cytokine with a ligand or cell may be conducted under acidic conditions, wherein, for example, the pH is, for example less than about 7.5 to about 7.2, for example less than about pH7.4 or pH7.3. Alternatively, the step of contacting the modified cytokine with a ligand or cell may be conducted at a pH of between about 4.0 and about 7.0. The step of contacting the modified cytokine with a ligand or cell may be conducted at a pH of between about 4.5 or about pH 4.8 to about pH 5.5 or pH 6.5 or from about pH 5.0 to about pH 6.9, for ex ample at a pH of about 5.1, about 5.2, about 5.3, about 5.4, about 5.5, about 5.6, about 5.7, about 5.8, about 5.9, about 6.0, about 6.1, about 6.2 and about 6.3 or about 6.4.

Any step of determining whether or not a modified cytokine activates a cell may comprise contacting the cell with the modified cytokine and detecting, for example, proliferation and/or expansion of the cell, upregulated expression of cell surface markers and/or the expression of other cytokines or molecules from the cell.

Useful cytokine muteins may be identified using a directed evolution approach/iterative selection cycles in which decreasing concentrations of cytokine receptor/ligand are contacted with the cytokine muteins. This helps identify cytokine muteins with the best receptor binding affinity.

Iterative selection cycles may first comprise binding (via one or several cycles) to a cytokine receptor multimer, preferably a receptor tetramer, and subsequently binding (in one or several cycles) to a cytokine receptor monomer. The receptor multimers may, for example, be obtained by binding biotinylated receptors to streptavidin or via other ligand/binder interactions.

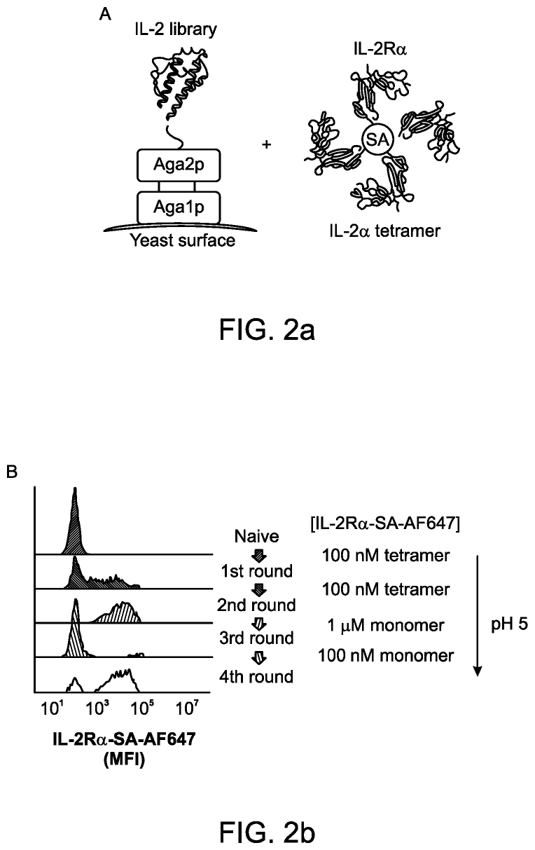

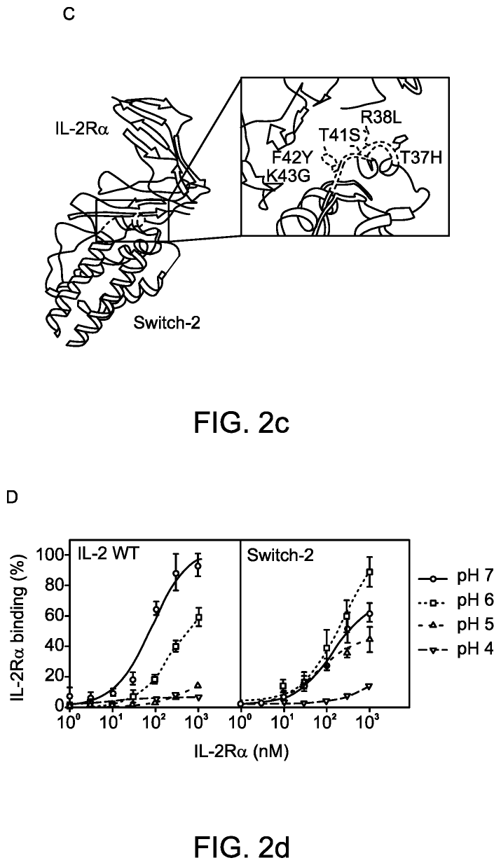

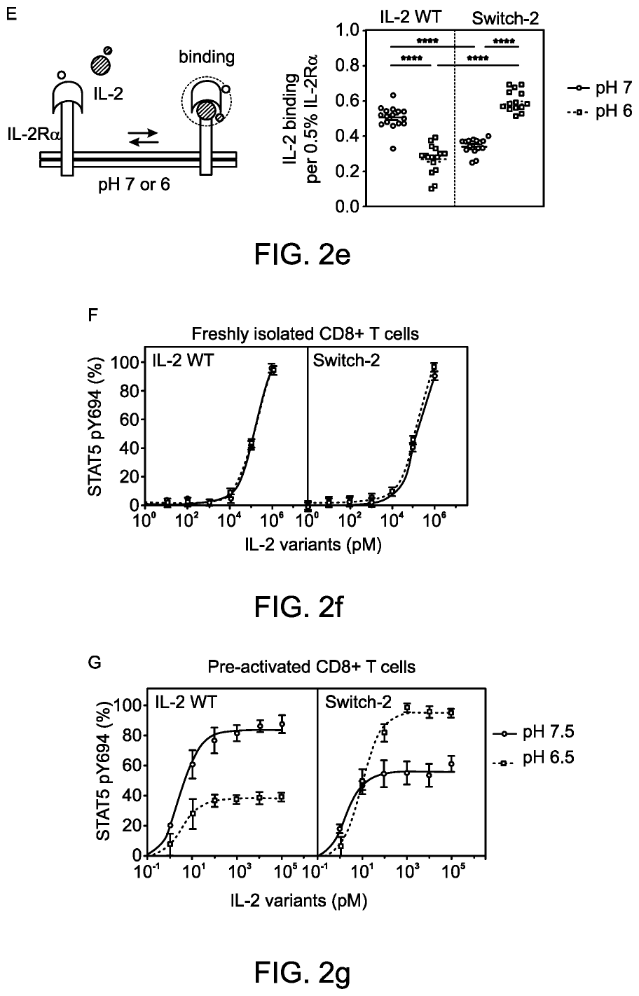

The iterative selection rounds may comprise binding under decreasing receptor concentration (for example 100 nM tetramer, 1 μM tetramer, 100 nM monomer; see also FIG. 2b). Without wishing to be bound by theory, decreasing the receptor concentration helps to identify mutein(s) with increasing receptor affinity.

Cytokine mutein(s) which are found to bind to the receptor in the various selection rounds may then be expressed using an expression vector/vehicle comprising a nucleic acid encoding the relevant mutein(s). Accordingly, the method may further comprise a step of isolating and/or sequencing the nucleic acids comprised in the expression vehicle(s)/vector(s) bound to a receptor via the expressed mutein(s).

The method may further comprise a step of contacting cytokine mutein(s) with a corresponding receptor or binding fragment thereof at a pH of at least 7.2, preferably about 7.4. Additionally, the method may comprise contacting the corresponding wild-type cytokine with said corresponding receptor or binding fragment thereof. Using either or both these method steps, the user may be able to determine the binding affinities of the cytokine mutein(s) and wild-type cytokine under the respective conditions. The method may further comprise a step of selecting mutein(s) which bind the corresponding receptor at the respective pH with a lower affinity compared to the wild-type cytokine.

The method may further comprise a step of selecting cytokine mutein(s) which bind to their corresponding receptor or binding fragment thereof at a pH of between about 4.0 and about 7.0 with a higher affinity as compared to pH of at least 7.2, preferably about 7.4. Preferably, at this step muteins may be selected which further are characterized by binding to the corresponding receptor at pH of at least 7.2, preferably about 7.4, with a lower affinity as compared to the wild-type cytokine.

The invention further relates to a library comprising nucleic acids encoding cytokine muteins and a cytokine mutein library as described above.

The techniques described herein may be applied to interleukin-2 (IL-2) which drives T cell expansion and regulates various effector functions. IL-2 induces cytotoxic functions, including, for example, the production of IFNγ. Crucial to IL-2 function is its binding activity. IL-2 receptors include, for example, IL2Rα, IL2Rβ and IL2Rγ. For convenience, these receptors will be collectively referred to as “IL-2 receptors”.

IL-2 has been used as an immunotherapy for malignancies. However, some of the crucial functions of IL-2 are sensitive to pH changes; not least, binding between IL-2 and its receptors is a pH sensitive process. Without being bound by theory, the acid pH found in the TME inhibits IL-2 responses by blocking its binding to, for example, IL-2Rα. The acidic tumour microenvironment (TME) adversely influences IL-2 receptor binding and affects IL-2 signalling. In turn, this results in weak STAT5 activation by IL-2 in the tumour and reduced IFNγ/TNFα secretion by CD8+ T cells. Combined this has the potential to reduce the efficacy of any IL-2 based therapeutic—especially when used to treat a cancer.

The present disclosure provides IL-2 muteins, which are pH resistant and retain crucial therapeutic functions at an acidic extracellular pH. Moreover, certain therapeutic functions assigned to these IL-2 muteins are more potent or effective at an acidic pH than they are at a neutral or other pH. Without being bound by theory, this has the advantage of making the IL-2 muteins described herein selective to the treatment of diseased cells/tissues and especially those that induce or create an acidic microenvironment.

The IL-2 muteins of this disclosure:
bind to any one of the disclosed IL-2 receptors; and/or
bind to IL-2Rα; and/or
bind to an IL-2 receptor or to IL-2Ra with higher affinity at a pH selected from a pH of about 4.0 to about 7.0, preferably about 5 to about 6.5, than at a pH selected from a pH of about 7.2 to about 7.5.; and/or
bind to IL-2 receptor or IL-2Ra with a lower affinity at a pH of about 7.2 to about 7.5 compared to a wild-type IL-2 molecule; and/or
bind to an IL-2 receptor or to IL-2Ra with higher affinity at a pH selected from a pH of about 4.0 to about 7.0, preferably about 5 to about 6.5, compared to a wild-type IL-2 molecule; and or
trigger STAT5 activation; and/or
trigger more potent STAT5 activation at pH 6.5 than at pH 7.2.

The IL-2 muteins according to this disclosure bind to IL-2 receptors (including for example, IL-2Rα) with higher affinity at a pH selected from a pH of about 4.0 to about 7.0, wherein that binding with higher affinity at a pH of about 4.0 to about 7.0 is characterized by a binding constant Kd which is about 0.3; about 0.5; about 0.8; about 1; about 1.5; about 2; about 2.5 or about 3 orders of magnitude lower than the binding constant Kd for the binding at a pH of about 7.2 to about 7.5.

Furthermore, the binding of the IL-2 mutein to an IL-2 receptor (for example, IL-2Rα) with a lower affinity at a pH of about 7.2 to about 7.5 compared to a wild-type IL-2 molecule may be characterized by a binding constant Kd which is about 0.3; about 0.5; about 0.8; about 1; about 1.5; about 2; about 2.5 or about 3 orders of magnitude higher for the IL-2 mutein as compared for the wild-type IL-2 molecule.

The binding of the IL-2 mutein to an IL-2 receptor (for example, IL-2Rα) with higher affinity at a pH selected from a pH of about 4.0 to about 7.0, compared to a wild-type IL-2 molecule, may be characterized by a binding constant Kd which is about 0.3; about 0.5; about 0.8; about 1; about 1.5; about 2; about 2.5 or about 3 orders of magnitude lower for the IL-2 mutein as compared to wild-type IL-2 molecule.

Without wishing to be bound by theory, the binding between a IL-2 muteins and its high affinity receptor complex may trigger more potent STAT5 phosphorylation at pH6.5 than at pH 7.2 by stabilizing the cytokine and the cytokine receptor complex. Moreover (and again without being bound by theory) an IL-2 mutein may induce superior expansion of activated T cells expressing a high affinity receptor complex in an acidic micro environment such as that found in the tumor micro environment (TME) and tertiary lymphoid structures (TLS).

Due to the higher activity of the IL-2 muteins according to the invention in the tumour micro environment (TME) and tertiary lymphoid structures (TLS) and a comparably lower activity in the periphery, such as in blood, the IL-2 muteins according to the invention may overcome the problems of dose limiting toxicity which is associated with prior art IL-2 therapies. Furthermore, when used in combination with other therapeutic molecules, for example antibodies against checkpoint inhibitors, the action of prior art IL-2 molecules limits the dose of such other molecules due to combined toxicity in the periphery. Accordingly, the selective activity of the IL-2 mutein may reduce toxicity in a combination treatment and may allow for higher doses of other therapeutic molecules, for example antibodies against checkpoint inhibitors, and may thus increase the therapeutic effect of such treatments. Combination treatments (comprising a cytokine mutein, IL-2 mutein of this disclosure and some other therapeutic/active agent(s) are described elsewhere in this specification).

It should be noted that the terms “comprise”, “comprising” and/or “comprises” is/are used to denote that aspects and embodiments of this invention “comprise” a particular feature or features. It should be understood that this/these terms may also encompass aspects and/or embodiments which “consist essentially of” or “consist of” the relevant feature or features.

Relative to a wild-type or reference IL-2 sequence, the IL-2 muteins of this disclosure are modified. For example, relative to a wild-type or reference sequence, the IL-2 muteins of this disclosure comprise one or more amino acid modifications. An amino acid modification may comprise the substitution of a wild-type or reference amino acid with another. Such substitutions may be conservative in that they swap a wild-type residue for another with the same or similar structural, chemical and/or physio-chemical properties. “Conservative” amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

Substitutions may also be ‘non-conservative’ in that a wild-type residue is substituted for an amino acid of a different class, for example an amino acid which is structurally dissimilar, chemically different and/or physio-chemically different or dissimilar.

An amino acid modification may comprise the deletion of an amino acid residue from a wild-type or reference sequence. Other amino acid modifications may comprise the insertion of one or more amino acids into a wild-type/reference sequence. Amino acid modifications may further comprise the inversion of certain parts or portions of the wild-type/reference sequence.

A IL-2 mutein of this disclosure may comprise (relative to a wild-type or reference sequence) one or more of these modifications, for example, one or more (e.g. 2, 3, 4, 5, 6 or more) amino acid substitutions, the deletion of one or more (for example 2, 3, 4, 5, 6 or more) amino acid residues and/or the addition of one or more (for example 2, 3, 4, 5, 6 or more) amino acid residues. A modified sequence may further comprise the inversion of one or more (for example 2, 3, 4, 5, 6 or more) parts of the wild type or reference sequence.

A reference or wild-type IL-2 sequence may comprise the human mature IL-2 sequence which is represented here by SEQ ID NO: 1.

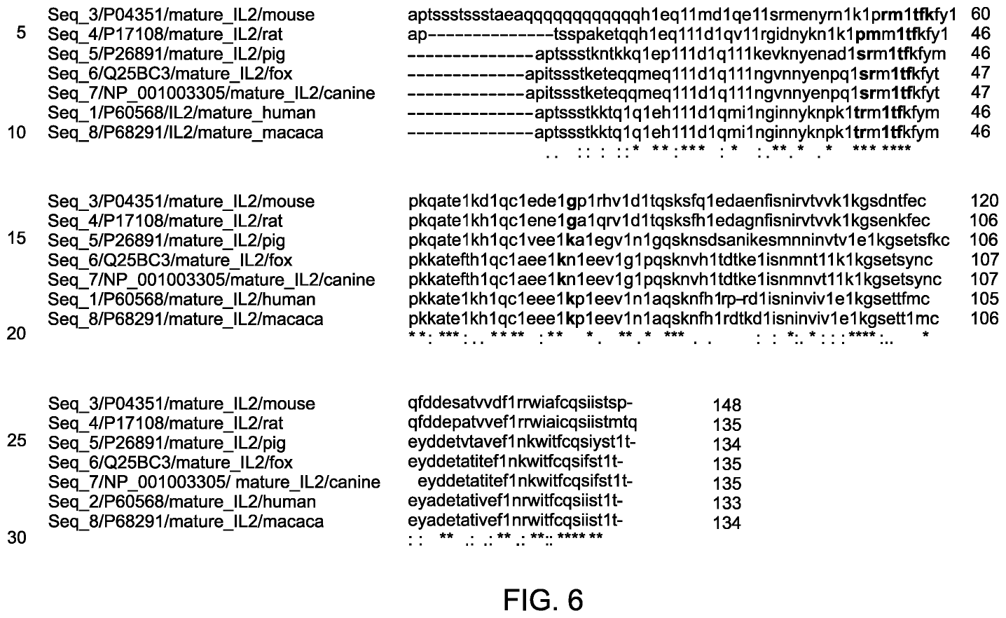

As evident from the sequence alignment depicted in FIG. 6, the IL-2 sequence is highly conserved over various mammalian species. Accordingly, in alternative embodiments the wild-type IL-2 sequence may comprise the mature IL-2 sequence from mouse (SEQ ID NO: 3), rat (SEQ ID NO: 4), pig (SEQ ID NO: 5), fox (SEQ ID NO: 6), dog (SEQ ID NO: 7), or macaca (SEQ ID NO: 8) disclosed in Table 1:

Table 1: WT IL-2 sequences

TABLE 1

| SEQ | | |
|---|---|---|
| ID NO: | Species | Sequence |
| 1 | human | APTSSSTKKT QLQLEHLLLD LQMILNGINN |
| | | YKNPKLTRML TFKFYMPKKA TELKHLQCLE |
| | | EELKPLEEVL NLAQSKNFHL RPRDLISNIN VIVLELKGSE |
| | | TTFMCEYADE TATIVEFLNR WITFCQSIIS |
| 3 | mouse | APTSSSTSSS TAEAQQQQQ QQQQQQHLEQ |
| | | LLMDLQELLS RMENYRNLKL PRMLTFKFYL |
| | | PKQATELKDL QCLEDELGPL RHVLDLTQSK |
| | | SFQLEDAENF ISNIRVTVVK LKGSDNTFEC |
| | | QFDDESATVV DFLRRWIAFC QSIISTSP |
| 4 | rat | APTSSPAKET QQHLEQLLLD LQVLLRGIDN |
| | | YKNLKLPMML TFKFYLPKQA TELKHLQCLE |
| | | NELGALQRVL DLTQSKSFHL EDAGNFISNI |
| | | RVTVVKLKGS ENKFECQFDD EPATVVEFLR |
| | | RWIAICQSII STMTQ |
| 5 | pig | APTSSSTKNT KKQLEPLLLD LQLLLKEVKN |
| | | YENADLSRML TFKFYMPKQA TELKHLQCLV |
| | | EELKALEGVL NLGQSKNSDS ANIKESMNNI |
| | | NVTVLELKGS ETSFKCEYDD ETVTAVEFLN |
| | | KWITFCQSIY STLT |
| 6 | fox | APITSSSTKE TEQQMEQLLL DLQLLLNGVN |
| | | NYENPQLSRM LTFKFYTPKK ATEFTHLQCL |
| | | AEELKNLEEV LGLPQSKNVH LTDTKELISN |
| | | MNVTLLKLKG SETSYNCEYD DETATITEFL |
| | | NKWITFCQSI FSTLT |
| 7 | dog | APITSSSTKE TEQQMEQLLL DLQLLLNGVN |
| | | NYENPQLSRM LTFKFYTPKK ATEFTHLQCL |
| | | AEELKNLEEV LGLPQSKNVH LTDTKELISN |
| | | MNVTLLKLKG SETSYNCEYD DETATITEFL |
| | | NKWITFCQSI FSTLT |
| 8 | macaca | APTSSSTKKT QLQLEHLLLD LQMILNGINN |
| | | YKNPKLTRML TFKFYMPKKA TELKHLQCLE |
| | | EELKPLEEVL NLAQSKNFHL RDTKDLISNI |
| | | NVIVLELKGS ETTLMCEYAD ETATIVEFLN RWITFCQSII |
| | | STLT |

In view of the above, a modified IL-2 molecule or IL-2 mutein according to this disclosure may, relative to the sequence of SEQ ID NO: 1, 2 to 8 comprise one or more amino acid modification(s).

In one teaching, the one or more amino acid modification(s) are selected from:
(i) one or more amino acid substitutions (including, for example, conservative substitutions);
(ii) one or more amino acid deletions;
(iii) one or more amino acid additions; and
(iv) one or more sequence inversions.

A modified IL-2 molecule or IL-2 mutein may comprise a mutation at any one or more residues selected from residue 35 to residue 45, residue 58 to residue 71 and/or residue 107 to residue 112 of SEQ ID NO: 1, 4, 5, 8 or respective residues in SEQ ID NO: 3, 6, or 7. In one teaching, a modified IL-2 molecule or IL-2 mutein may comprise a mutation at any one or more of the residues from residue 37 to residue 43, residue 60 to residue 69, residue 109 to residue 110 of SEQ ID NO: 1, 4, 5, or 8 or respective residues in SEQ ID NO: 3, 6, or 7. A modified IL-2 molecule or IL-2 mutein may comprise a mutation at any one or more of residue 37, residue 38, residue 41, residue 42, residue 43, residue 60, residue 61, residue 63, residue 64, residue 66, residue 68, residue 69, residue 109, and residue 110 of SEQ ID NO: 1, 4, 5, or 8 or respective residues in SEQ ID NO: 3, 6, or 7. A modified IL-2 molecule or IL-2 mutein may comprise a mutation at any one or more of residue 37, residue 38, residue 41, residue 42, residue 43, and residue 64 of SEQ ID NO: 1, 4, 5, or 8 or respective residues in SEQ ID NO: 3, 6, or 7.

The term “respective residue” defines which residues in SEQ ID NO: 3, 6, or 7 correspond to which residues in SEQ ID NO: 1, 4, 5, 8—this is based on the sequence alignments as follows:

TABLE 2

| Residue in SEQ | respective | respective |
|---|---|---|
| ID NO: 1, 4, 5, | residue” in SEQ | residue” in SEQ |
| 8 | ID NO: 6, 7 | ID NO: 3 |
| 35 | 36 | 49 |
| 37 | 38 | 51 |
| 38 | 39 | 52 |
| 41 | 42 | 55 |
| 42 | 43 | 56 |
| 43 | 44 | 57 |
| 45 | 46 | 59 |
| 58 | 59 | 72 |
| 60 | 61 | 74 |
| 61 | 62 | 75 |
| 63 | 64 | 77 |
| 64 | 64 | 78 |
| 66 | 67 | 80 |
| 68 | 69 | 82 |
| 69 | 70 | 83 |
| 71 | 72 | 85 |
| 109 | 110 | 123 |
| 110 | 111 | 124 |

This disclosure provides an IL-2 mutein, comprising (relative to SEQ ID NO: 1, 4, 5, or 8 or respective residues of SEQ ID NO: 3, 6, or 7 or a wild-type/reference sequence) an amino acid substitution at residue 37. By way of example, an IL-2 mutein of this disclosure may comprise a threonine to histidine, arginine or serine substitution at residue 37. In one teaching and in addition to an amino acid modification at position 37, an IL-2 mutein may further comprise one or more modifications at one or more other residues. For example, an IL-2 mutein may comprise an amino acid modification at residue 37 and one or more additional amino acid modifications at any of positions 38, 41, 42, 43 and/or 64.

This disclosure provides an IL-2 mutein, comprising (relative to SEQ ID NO: 1, 4, 5, or 8 or a wild-type/reference sequence) an amino acid substitution at residue 38. By way of example, an IL-2 mutein of this disclosure may comprise an arginine to leucine, valine, isoleucine or alanine substitution at residue 38. In one teaching and in addition to an amino acid modification at position 38, an IL-2 mutein may further comprise one or more modifications at one or more other residues. For example, an IL-2 mutein may comprise an amino acid modification at residue 38 and one or more additional amino acid modifications at any of positions 37, 41, 42, 43 and/or 64. Likewise, the same embodiments are provided based on SEQ ID NO: 3, 6, or 7 and the respective residues in SEQ ID NO: 3, 6, or 7.

This disclosure provides an IL-2 mutein, comprising (relative to SEQ ID NO: 1, 4, 5, or 8 or a wild-type/reference sequence) an amino acid substitution at residue 41. By way of example, an IL-2 mutein of this disclosure may comprise a threonine to serine, glycine, or aspartic acid substitution at residue 41. In one teaching and in addition to an amino acid modification at position 41, an IL-2 mutein may further comprise one or more modifications at one or more other residues. For example, an IL-2 mutein may comprise an amino acid modification at residue 41 and one or more additional amino acid modifications at any of positions 37, 38, 42, 43 and/or 64. Likewise, the same embodiments are provided based on SEQ ID NO: 3, 6, or 7 and the respective residues in SEQ ID NO: 3, 6, or 7.

This disclosure provides an IL-2 mutein, comprising (relative to SEQ ID NO: 1, 4, 5, or 8 or a wild-type/reference sequence) an amino acid substitution at residue 42. By way of example, an IL-2 mutein of this disclosure may comprise a phenylalanine to tyrosine substitution at residue 42. In one teaching and in addition to an amino acid modification at position 42, an IL-2 mutein may further comprise one or more modifications at one or more other residues. For example, an IL-2 mutein may comprise an amino acid modification at residue 42 and one or more additional amino acid modifications at any of positions 37, 38, 41, 43 and/or 64. Likewise, the same embodiments are provided based on SEQ ID NO: 3, 6, or 7 and the respective residues in SEQ ID NO: 3, 6, or 7.

This disclosure provides an IL-2 mutein, comprising (relative to SEQ ID NO: 1, 4, 5, or 8 or a wild-type/reference sequence) an amino acid substitution at residue 43. By way of example, an IL-2 mutein of this disclosure may comprise a lysine to glycine substitution at residue 43. In one teaching and in addition to an amino acid modification at position 43, an IL-2 mutein may further comprise one or more modifications at one or more other residues. For example, an IL-2 mutein may comprise an amino acid modification at residue 43 and one or more additional amino acid modifications at any of positions 37, 38, 41, 42 and/or 64.

This disclosure provides an IL-2 mutein, comprising (relative to SEQ ID NO: 1, 4, 5, or 8 or a wild-type/reference sequence) an amino acid substitution at residue 64. By way of example, an IL-2 mutein of this disclosure may comprise a non-conservative amino acid substitution of lysine, preferably a substitution with an acidic amino acid, most preferably to glutamic acid substitution at residue 64. In one teaching and in addition to an amino acid modification at position 64, an IL-2 mutein may further comprise one or more modifications at one or more other residues. For example, an IL-2 mutein may comprise an amino acid modification at residue 64 and one or more additional amino acid modifications at any of positions 37, 38, 41, 42 and/or 43.

By way of a summary, the disclosure embraces the following IL-2 muteins:

TABLE 3

| WT | | Switch2 | MUT1 | IL2 B1 | IL2 B11 | IL2 B2 | IL2 2A |
|---|---|---|---|---|---|---|---|
| 37 | T | H | S | S | S | R | S |
| 38 | R | L | A | L | V | V | I |
| 41 | T | S | D | G | G | G | G |
| 42 | F | Y | | Y | | | |
| 43 | K | G | G | G | G | G | G |
| 64 | K | K | E | | | | |

This disclosure provides an IL-2 mutein, comprising at least a modification at positions 37, 38, 41, 43 and at least one further modification at positions 42 or 64 of SEQ ID NO: 1, 4, 5, 8 or respective residues in SEQ ID NO: 3, 6, or 7.

This disclosure provides an IL-2 mutein, comprising amino acid modifications to the each of the residues at positions 37, 38, 41, 43 and 64 of SEQ ID NO: 1, 4, 5, 8 or respective residues in SEQ ID NO: 3, 6, or 7.

An IL-2 mutein of this disclosure may comprise (relative to SEQ ID NO: 1 or 8 or a wild-type/reference sequence) a sequence characterised by one or more of the following amino acid mutations
(i) T37H; and/or
(ii) R38L; and/or
(iii) T41S; and/or
(iv) F42Y; and/or
(v) K43G.

Accordingly, an IL-2 mutein according to this disclosure may comprise SEQ ID NO: 2.

SEQ ID NO: 2
APTSSSTKKT QLQLEHLLLD LQMILNGINN YKNPKLHLML
SYGFYMPKKA TELKHLQCLE EELKPLEEVL NLAQSKNFHL
RPRDLISNIN VIVLELKGSE TTFMCEYADE TATIVEFLNR
WITFCQSIIS

An IL-2 mutein of this disclosure may comprise (relative to SEQ ID NO: 1 or 8 or a wild-type/reference sequence) a sequence characterised by one or more of the following amino acid mutations
(i) T37S; and/or
(ii) R38A; and/or
(iii) T41D; and/or
(iv) -K43G; and/or
(v) -K64E.

Accordingly, an IL-2 mutein according to this disclosure may comprise SEQ ID NO: 9.

SEQ ID NO: 9
APTSSSTKKT QLQLEHLLLD LQMILNGINN YKNPKLSAML
DFGFYMPKKA TELKHLQCLE EELEPLEEVL NLAQSKNFHL
RPRDLISNIN VIVLELKGSE TTFMCEYADE TATIVEFLNR
WITFCQSIIS

An IL-2 mutein of this disclosure may comprise (relative to SEQ ID NO: 1 or 8 or a wild-type/reference sequence) a sequence characterised by one or more of the following amino acid mutations
(i) T37S; and/or
(ii) R38L; and/or
(iii) T41G; and/or
(iv) -F42Y; and/or
(v) -K43G.

Accordingly, an IL-2 mutein according to this disclosure may comprise SEQ ID NO: 10.

SEQ ID NO: 10
APTSSSTKKT QLQLEHLLLD LOMILNGINN YKNPKLSLML
GYGFYMPKKA TELKHLQCLE EELKPLEEVL NLAQSKNFHL
RPRDLISNIN VIVLELKGSE TTFMCEYADE TATIVEFLNR
WITFCQSIIS

An IL-2 mutein of this disclosure may comprise (relative to SEQ ID NO: 1 or 8 or a wild-type/reference sequence) a sequence characterised by one or more of the following amino acid mutations
(i) T37S; and/or
(ii) R38V; and/or
(iii) T41G; and/or
(iv) K43G.

Accordingly, an IL-2 mutein according to this disclosure may comprise SEQ ID NO: 11.

SEQ ID NO: 11
APTSSSTKKT QLQLEHLLLD LQMILNGINN YKNPKLSVML
GFGFYMPKKA TELKHLQCLE EELKPLEEVL NLAQSKNFHL
RPRDLISNIN VIVLELKGSE TTFMCEYADE TATIVEFLNR
WITFCQSIIS

An IL-2 mutein of this disclosure may comprise (relative to SEQ ID NO: 1 or 8 or a wild-type/reference sequence) a sequence characterised by one or more of the following amino acid mutations
(i) T37R; and/or
(ii) R38V; and/or
(iii) T41G; and/or
(iv) K43G.

Accordingly, an IL-2 mutein according to this disclosure may comprise SEQ ID NO: 12.

SEQ ID NO: 12
APTSSSTKKT QLQLEHLLLD LQMILNGINN YKNPKLRVML
GFGFYMPKKA TELKHLQCLE EELKPLEEVL NLAQSKNFHL
RPRDLISNIN VIVLELKGSE TTFMCEYADE TATIVEFLNR
WITFCQSIIS

An IL-2 mutein of this disclosure may comprise (relative to SEQ ID NO: 1 or 8 or a wild-type/reference sequence) a sequence characterised by one or more of the following amino acid mutations
(i) T37S; and/or
(ii) R38I; and/or
(iii) T41G; and/or
(iv) K43G.

Accordingly, an IL-2 mutein according to this disclosure may comprise SEQ ID NO: 13.

SEQ ID NO: 13
APTSSSTKKT QLQLEHLLLD LQMILNGINN YKNPKLSIML
GFGFYMPKKA TELKHLQCLE EELKPLEEVL NLAQSKNFHL
RPRDLISNIN VIVLELKGSE TTFMCEYADE TATIVEFLNR
WITFCQSIIS

An IL-2 mutein may comprise a functional fragment of any of the modified molecules or muteins described herein. In one teaching, an IL-2 mutein of this disclosure may comprise, consist essentially of or consist of, a functional fragment of the sequence provided by SEQ ID NO: 2, 9, 10, 11, 12 or 13. A ‘functional’ fragment may include any fragment retaining one or more of the functions assigned to a (or the) larger/full or complete IL-2 mutein. For example, a fragment of this disclosure may retain one or more of the functions of a mutein which comprises the full sequence of SEQ ID NO: 2, 9, 10, 11, 12 or 13. Such functions may include, for example and ability bind to IL-2Rα; and/or an ability to bind to IL-2Ra with higher affinity at pH 6.5 than at pH 7.2; and/or an ability to trigger STAT5 activation; and/or and ability to trigger more potent STAT5 activation at pH 6.5 than at pH 7.2.

A mutein fragment may be tested for any given function (for example a binding function, T-cell activation function and/or immune effector function) using any number of different assays. By way of example, a binding assay may comprise contacting a test IL-2 mutein fragment with IL-2Rα; in such an assay, the detection of binding between the fragment and IL-2Ra indicates that the fragment retains the necessary binding function. Furthermore, a fragment may be tested for an ability to drive T cell expansion and/or immune effector functions, using an assay which brings the fragment into contact with CD8+ T cells. Functional fragments will stimulate the CD8+ T cells to expand and/or to produce effector cytokines. Any binding, T-cell activation and/or effector function assays may be conducted at an acidic pH, for example a pH of less than PH7.4, for example a pH of about 6.1, about 6.2, about 6.3, about 6.4, about 6.5 (or at any other pH described herein). Not only would this test the functional capability of the fragments, but it would also determine whether or not the fragments retain the feature of being acid resistant. The results of these assays may be compared to, for example, the results of positive and/or control assays which use wild-type IL-2, IL-2 mutein(s) and/or IL-2 (mutein) fragments with known or predetermined functions. Control assays may also be conducted at different, for example neutral, pH in order to test for fragments that exhibit more potent activity/function at an acidic pH than at a neutral pH.

A fragment of SEQ ID NO: 2, 9, 10, 11, 12 or 13 may comprise anywhere between about 10 and about n−1 residues (where n=130; i.e. the total number of residues in SEQ ID NO: 2, 9, 10, 11, 12 or 13). By way of example, a fragment may comprise 15, 20, 25, 30, 35, 40, 45, 50, 55, 60, 65, 70, 75, 80, 85, 90, 95,100,105, 110, 115, 120, 125 or 129 amino acid residues of SEQ ID NO: 2, 9, 10, 11, 12 or 13.

A fragment of SEQ ID NO:2 may at least comprise residue numbers 57, 58, 61, 62 and 63. In one teaching a fragment of SEQ ID NO: 2, 9, 10, 11, 12 or 13 may comprise residues 57-63. A fragment comprising any of these select residues may further comprise fragments of the sequences which lie immediately up and/or downstream thereof.

An IL-2 mutein of this disclosure may further exhibit a level of sequence identity or homology with the sequence of SEQ ID NO: 2, 9, 10, 11, 12 or 13. For example, useful fragments may have a sequence which is at least 60%, 65%, 70%, 75%, 80%, 85%, 90%, 95%, 96%, 97%, 98% or 99% identical or homologous to the sequence of SEQ ID NO: 2, 9, 10, 11, 12 or 13.

In view of the above, the term IL-2 mutein embraces not only the specific example provided by SEQ ID NO: 2, 9, 10, 11, 12 or 13, but functional fragments thereof, molecules with some level of sequence identity/homology to SEQ ID NO: 2, 9, 10, 11, 12, 13 and/or other IL-2 derived molecules comprising one or more of the described amino acid modifications.

This disclosure further provides a nucleic acid encoding any of the modified IL-2 mutein(s) described herein. For example, the disclosure provides a nucleic acid encoding an IL-2 mutein which, relative to a reference or wild-type sequence, has one or more amino acid modifications. The nucleic acid may be a DNA, RNA, preferably an mRNA.

The disclosure provides nucleic acids which encode SEQ ID NO: 2, 9, 10, 11, 12, 13 or any functional fragment thereof.

A nucleic acid of this disclosure may be codon optimised for expression in a host cell, for example a microbial host cell.

Disclosed herein is a vector comprising a nucleic acid of this disclosure. For example, the disclosure provides a vector comprising a nucleic acid which encodes an IL-2 mutein, SEQ ID NO: 2, 9, 10, 11, 12, 13 or a functional fragment thereof.

Also disclosed is a host cell transformed with a nucleic acid or vector of this disclosure. The host cell may be a eukaryotic or a prokaryotic cell. The host cell may be a mammalian cell, an insect cell or a plant cell. The host cell may be a microbial cell—for example a bacteria (E. coli or the like). The host cell may be a T-cell, preferably a T cell comprising a chimeric antigen receptor (CAR).

Also disclosed herein is a virus comprising a nucleic acid of this disclosure.

A method of making a mutein of this disclosure may comprise transforming a host cell with an IL-2 mutein encoding nucleic acid or vector of this disclosure and inducing expression of the IL-2 mutein encoding nucleic acid. In such a method, the expressed IL-2 mutein can be harvested, extracted or purified from the host cell or from the medium in which the host cell is cultured.

This disclosure also provides a method of identifying a pH-resistant IL-2 mutein, said method comprising:
mutating or modifying a IL-2 molecule to generate an IL-2 mutein; and contacting the IL-2 mutein with IL-2Ra under acidic conditions so as to identify mutein(s) which bind IL-2Ra and are pH resistant.

The IL-2 molecule which is to be mutated or modified may comprise a wild-type IL-2 sequence or some other IL-2 reference sequence. For example, the IL-2 molecule to be modified or mutated may comprise SEQ ID NO: 1 or a functional fragment thereof.

The step of mutating or modifying an IL-2 molecule to generate an IL-2 mutein may comprise introducing one or more amino acid modifications into the wild-type or reference IL-2 sequence. Various techniques may be used to achieve this, including, for example, PCR based methods which exploit the use of primers comprising degenerate (for example NDT) codons to randomly mutate specific residues within the wild-type IL-2 sequence. Any of the muteins can be tested for an ability to bind IL-2Rα.

The step of contacting the modified IL-2 (IL-2 mutein) with IL-2Rα, may comprise contacting the modified IL-2 with an IL-2Ra fragment, wherein the IL-2Ra fragment is an IL-2 binding fragment. The IL-2 binding fragment of the IL-2Ra may comprise an ectodomain. The IL-2Ra or any binding fragment thereof may be conjugated to a binding moiety, such as, for example, biotin. The IL-2Ra may comprise a detectable label, for example a fluorescent label for identification of mutein(s) bound to a receptor.

A method of identifying a pH-resistant IL-2 mutein may further comprise a step of generating a library comprising nucleic acids encoding IL-2 muteins or fragments thereof, wherein the IL-2 muteins comprise one or more amino acid substitutions (including, for example, conservative substitutions); (ii) one or more amino acid deletions; (iii) one or more amino acid additions; and (iv) one or more sequence inversions (all of which are described/defined later in this specification).

The step of mutating or modifying an IL-2 molecule to generate an IL-2 mutein may comprise introducing a mutation (e.g. a substitution, an addition, a deletion or inversion) at any one or more residues from residue 35 to residue 45, residue 58 to residue 71 and/or residue 107 to residue 112 of SEQ ID: NO 1. In one teaching, the step of mutating or modifying an IL-2 molecule to generate an IL-2 mutein may comprise introducing a mutation at any one or more of the residues from residue 37 to residue 43, residue 60 to residue 69, residue 109 to residue 110 of SEQ ID: NO 1. The step of mutating or modifying an IL-2 molecule to generate an IL-2 mutein may comprise introducing a mutation at any one or more of residue 37, residue 38, residue 41, residue 42, residue 43, residue 60, residue 61, residue 63, residue 64, residue 66, residue 68, residue 69, residue 109, and residue 110 of SEQ ID: NO 1. The step of mutating or modifying an IL-2 molecule to generate an IL-2 mutein may comprise introducing a mutation at any one or more of residue 37, residue 38, residue 41, residue 42, and residue 43 of SEQ ID: NO 1.

The nucleic acids of any created library may encode muteins which comprise a substitution of the natural or wild type amino acid for any other amino acid; for example, substitution of the natural or wild type amino acids with another amino acid selected from amino acids G, V, L, I, C, A, E, S, R, H, D, N, F, and Y. A nucleic acid library may be generated by nested PCR using primers with degenerated codons for any one or more of the aforementioned amino acids.

The method may further comprise a step of expressing the nucleic acid library to obtain a IL-2 mutein library. The IL-2 mutein(s) comprised in the library may be expressed on the surface of an expression vehicle such as a cell, a virus of a phage, for example a yeast cell.

The step of contacting the IL-2 mutein with IL-2Ra may be conducted at a range of different pHs. For example, the step of contacting the IL-2 mutein with IL-2Ra may be conducted at a pH which is less than about pH 7.4. For example, the contacting step may be conducted at pH of between about pH 4.0 or pH 5.0 and about pH 7.0 or pH7.3. For example, the contacting step may be conducted at a pH of between about pH 4.5 or pH 4.8 and about pH 6.0 or pH 6.5. The contacting step may be conducted at pH 5.5, pH 6.0, pH 6.1, pH 6.2, pH 6.3, pH 6.4, pH 6.5, pH 6.6, pH 6.7, pH 6.8, pH 6.9, pH 7.0, pH 7.1 or pH 7.2. In a method of this type, a mutein which is found to bind IL-2Ra at an acidic pH can be identified as an IL-2 mutein which may be acid resistant. Additionally or alternatively, an IL-2 mutein exhibiting stronger receptor (IL-2Rα) binding at an acidic pH as compared to the respective wild-type, may be identified as an IL-2 mutein which may be acid resistant.

Useful IL-2 muteins may be identified using a directed evolution approach/iterative selection cycles in which decreasing concentrations of IL-2Ra (for example IL-2Ra ectodomain) are contacted with IL-2 muteins. This helps identify IL-2 muteins with the best receptor binding affinity.

Iterative selection cycles may first comprise binding (via one or several cycles) to a cytokine receptor multimer, preferably a receptor tetramer, and subsequently binding (in one or several cycles) to a cytokine receptor monomer. The receptor multimers may, for example, be obtained by binding biotinylated receptors to streptavidin or via other ligand/binder interactions.

The iterative selection rounds may comprise binding under decreasing receptor concentration (for example 100 nM tetramer, 1 μM tetramer, 100 nM monomer; see also FIG. 2b). Without wishing to be bound by theory, decreasing the receptor concentration helps to identify mutein(s) with increasing receptor affinity.

IL-2 mutein(s) bound to the receptor and identified in the selection rounds may then be expressed using an expression vehicle/vector comprising a nucleic acid encoding the relevant mutein(s). Accordingly, the method may further comprise a step of isolating and/or sequencing the nucleic acids comprised in the expression vehicle(s) bound to a receptor via the expressed mutein(s).

The method may further comprise a step of contacting the identified IL-2 mutein with a corresponding receptor or binding fragment thereof under a pH of at least 7.2, preferably about 7.4 so as to identify mutein(s) which bind the corresponding receptor at the respective pH with a lower affinity compared to the wild-type cytokine.

The invention further relates to a library comprising nucleic acids encoding cytokine muteins and cytokine mutein library as described above.

Acid resistant IL-2 muteins may find application in the treatment and/or prevention of range of different diseases and/or conditions, including cancer.

As such, this disclosure provides a method of identifying an IL-2 mutein for use in the treatment of cancer, said method comprising:
mutating or modifying an IL-2 molecule to generate an IL-2 mutein; and contacting the IL-2 mutein with IL-2Ra to identify muteins which bind IL-2Rα.

Again, the step of mutating or modifying an IL-2 molecule to generate an IL-2 mutein may comprise introducing one or more amino acid modifications into a wild-type or reference TL-2 sequence. Additionally, the step of contacting the TL-2 mutein with IL-2Ra may be conducted at an acidic pH. The aim being to identify IL-2 muteins which are able to bind IL-2Ra at an acidic pH. Since the tumour microenvironment may be acidic, IL-2 muteins which are pH resistant maybe most useful in the treatment and/or prevention of cancer.

This disclosure also provides a pH-resistant IL-2 mutein obtainable by a method comprising mutating or modifying an IL-2 molecule to generate an IL-2 mutein; and contacting the IL-2 mutein with IL-2Ra under acidic conditions so as to identify those mutein(s) which bind IL-2Ra and are therefore pH resistant. The IL-2 molecule to be mutated or modified may comprise a wild-type IL-2 sequence or some other IL-2 reference sequence. For example, the IL-2 molecule to be modified may comprise SEQ ID NO: 1 or a functional fragment thereof. The acidic conditions may be formulated as described above. Muteins which retain an ability to bind IL-2Ra under acidic conditions may be very useful as agents for use in the treatment of cancer where the TME is acidic and inhibits the function of standard IL-2-based therapeutics. Preferably, the disclosed IL-2 muteins and fusion proteins are for use in the treatment of a cancer with an extracellular pH (pHe) of the TME in a tumour of said cancer of lower than about pH 7.4, lower than 7.2, preferably lower than 7.0, preferably lower than about 6.8, most preferably lower than about 6.6. Such cancer types may for example be lymphoid cancers or solid cancers. The treatment of a cancer may comprise a step of determining the extracellular pH of the TME in a patient prior to administering an IL-2 mutein, fusion protein or related composition disclosed herein. The pHe of the TME may be determined according to various methods known in the art including fluorescence imaging, PET, ¹H Magnetic resonance spectroscopy (MRS), ³¹P MRS, ¹⁹F MRS, Hyperpolarized ¹³C MRS, Magnetic resonance imaging (MRI), especially CEST MRI as disclosed by Chen²² (the entire contents of the disclosures being incorporated herein by reference). Preferably the pHe of the TME is determined by MRI.

This disclosure further provides modified IL-2 molecules for use in methods, compositions and medicaments for the treatment and/or prevention of a range of diseases and/or conditions.

Accordingly, the disclosure provides any of the IL-2 mutein(s) for use in medicine.

In one teaching, the disclosure provides a protein comprising SEQ ID NO: 2, 9, 10, 11, 12, 13 or a functional fragment thereof, for use in medicine. The definition of functional fragments is provided elsewhere in this specification.

The disclosure further provides a nucleic acid encoding any of the disclosed IL-2 mutein(s) for use in medicine. In one teaching, the disclosure provides a nucleic acid encoding a protein comprising SEQ ID NO: 2, 9, 10, 11, 12, 13 or a fragment thereof, for use in medicine.

The disclosure provides:
any of the disclosed IL-2 mutein(s); and/or
a protein comprising SEQ ID NO: 2, 9, 10, 11, 12, 13 or a fragment thereof; and/or
a nucleic acid encoding any of the disclosed IL-2 mutein(s); and/or
a nucleic acid encoding a protein comprising SEQ ID NO: 2, 9, 10, 11, 12, 13 or a fragment thereof;
for use as medicament.

In this regard, any of the IL-2 mutein(s) described herein may find application or use as an immunotherapy.

The disclosure provides a modified IL-2 molecule for use in the treatment or prevention of an immunological condition.

The disclosure provides a modified IL-2 molecule for use in the treatment or prevention of cancer.

In one teaching, the term ‘cancer’ includes cancers, the (tumour) cells of which are characterised by the over production of lactic acid. The term ‘cancer’ may also include any cancer yielding a tumour which creates an acidic microenvironment.

Also disclosed is the use of a modified IL-2 molecule of this disclosure in the manufacture of a medicament for the treatment or prevention of (i) a cancer, or (ii) an immunological condition.

The disclosure further provides a method of treating or preventing cancer, said method comprising administering a subject in need thereof a therapeutically effective amount of any of the modified IL-2 molecules described herein.

A subject to be administered a modified molecule of this disclosure may include any human or animal subject suffering from an immunological condition and/or a cancer. The subject may also be any human or animal subject predisposed and/or susceptible to an immunological condition or cancer, which cancer can be treated and/or prevented with the use of IL-2.

Without wishing to be bound by theory, a further advantage associated with the muteins of this disclosure is that they are less toxic than wild-type or unmodified IL-2 molecules. The muteins of this disclosure bind IL-2Ra with higher affinity at an acidic pH, trigger more potent STAT5 activation at pH 6.5 than at pH 7.2 and induce superior expansion of cytotoxic T cells as compared to a wild-type IL-2 molecule. As such, while high levels of systemic toxicity have hindered the therapeutic use of IL-2, the muteins provided by this exhibit reduced activity at neutral pH and are therefore selectively active within the acidic tumour microenvironment. In summary and again without being bound by theory, the IL-2 muteins of this disclosure induce potent responses within the acidic tumour microenvironment but are less likely to be systemically toxicity (than any wild-type (or unmodified) IL-2 molecule) due to reduced activity at neutral pH.

This disclosure may further provide a fusion protein comprising a cytokine mutein or IL-2 mutein described herein.

A fusion protein may further comprise one or more other molecule(s) bound, linked or fused to a cytokine mutein or IL-2 mutein. In one teaching the other molecule may be bound, linked or fused to the C-terminus, the N-terminus or the N- and C-terminus of the cytokine mutein or IL-2 mutein. In another teaching the fusion may comprise another molecule may be interested into the cytokine mutein or IL-2 mutein.

Accordingly, this disclosure provides a fusion protein comprising:
(i) a cytokine mutein; or
(ii) any of the disclosed IL-2 mutein(s); or
(iii) a protein comprising SEQ ID NO: 2, 9, 10, 11, 12, 13 or a fragment thereof.

The other molecule(s) of a fusion protein of this disclosure may comprise: a cytokine, cytokine mutein, or fragment thereof;
an interleukin molecule or fragment thereof
a polypeptide binding domain;
an antibody or a fragment thereof;
a single chain antibody;
a VHH.

A fusion protein of this disclosure may comprise an IL-2 mutein and at least one or more further different cytokine(s) as described herein.

A polypeptide binding domain for inclusion in a fusion protein of this disclosure may bind to or exhibit a specificity/affinity for a tumour antigen or a checkpoint molecule. Checkpoint molecules are negative regulators of immune responses, such as co-stimulatory receptors occurring on the surface of several immune cells and ligands to said receptors. The checkpoint molecule (to which a polypeptide binding molecule may bind or exhibit a specificity/affinity for) may be selected from CD27, CD137, 2B4, TIGIT, CD155, CD160, ICOS, HVEM, CD40L, LIGHT, LAIR1, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDO1, ID02, TDO, KIR, LAG-3, TIM-3, or VISTA.

As stated, a polypeptide binding domain for inclusion in a fusion of this invention may bind to or exhibit a specificity/affinity for a tumour antigen; the term ‘tumour antigen’ may embrace antigens selected from EpCAM, EGFR, HER-2, HER-3, c-Met, FoIR, PSMA, CD38, BCMA, CEA, 5T4, AFP, B7-H3, Cadherin-6, CAIX, CD117, CD123, CD138, CD166, CD19, CD20, CD205, CD22, CD30, CD33, CD40, CD352, CD37, CD44, CD52, CD56, CD70, CD71, CD74, CD79b, CLDN18.2, DLL3, EphA2, ED-B fibronectin, FAP, FGFR2, FGFR3, GPC3, gpA33, FLT-3, gpNMB, HPV-16 E6, HPV-16 E7, ITGA2, ITGA3, SLC39A6, MAGE, mesothelin, Mucd, Muc16, NaPi2b, Nectin-4, P-cadherin, NY-ESO-1, PRLR, PSCA, PTK7, ROR1, SLC44A4, SLTRK5, SLTRK6, STEAP1, TIM1, Trop2, or WT1. A polypeptide binding domain may bind a haematologic tumor antigen; a haematologic tumor antigen may be expressed by lymphoid cells. Tumour antigens of this type may include, for example, ADIR, AURKA, BCR-ABL, BMI1, CML28, CML66, Cyclin A1, DDX3Y, DKK1, FMOD, FRAME, G250/CAIX, HAGE, HM1.24, hTERT, LPP, MAG EA3, MAGEA3, MEF2D, MLL, MPP1, MUC1, Myeloperoxidase, NEWREN60, NY-ESO-1, PANE1, PRAME, Proteinase 3, PTPN20A/B, RHAMM, ROR1, SLAMF7, Survivin, TEX14, WT1, CD19, CD20, CD22, CD25, CD30, CD33, CD38, CD52, CD123, CD269, CD138, HM1.24, SLAMF7. The term (haematologic) tumour antigen may include, for example, surface antigens such as CD19, CD20, CD22, CD25, CD30, CD33, CD38, CD52, CD123, CD269, CD138, HM1.24, SLAMF7.

A polypeptide domain for use in a fusion of this disclosure may bind to, or exhibit an affinity/specificity for, an antigen expressed by a regulatory T-cell. An antigen expressed by a regulatory T cell may be comprised in a cell surface marker of a regulatory T-cell. The antigen expressed by a regulatory T cell may be selected from CTLA4, CD25, OX40, GITR, TNFRII, NRP1, TIGIT, CCR8, LAYN, MAGEH1, CD27, ICOS, LAG-3, TIM-3, CD30, IL-1R2, IL-21R, 4-1BB, PDL-1, and PDL-2.

A fusion protein of this disclosure may comprise an anti-Ox40 antibody or fragment thereof. Useful examples, may include those antibodies disclosed in WO 2015/132580A1 or US 2019275084 (the entire contents of these disclosures being incorporated herein by reference).

An antibody for use in a fusion protein of this disclosure may comprise an antagonistic antibody or an agonistic fragment thereof. A fragment of any of these antibodies may also be used, which fragments also exhibit the requisite antagonistic/agonistic activity. Useful agonistic antibodies may be disclosed in, for example, WO2020/006509, WO2018/045110, WO 2017/214092, WO2019/072868 (the relevant contents of all of these documents being incorporated herein).

An antibody for use in a fusion protein of this disclosure may comprise a pH sensitive antibody or a fragment thereof, wherein the fragment may retain the feature of being pH sensitive. A pH sensitive antibody will exhibit differential antigen binding kinetics at different pHs. For example and without wishing to be bound by theory, a pH sensitive antibody may, at an acidic pH, bind to an antigen with a higher or a lower affinity than it does to the same antigen at a different (e.g. neutral or alkali) pH. In one teaching, a pH sensitive antibody, may exhibit an increased affinity for an antigen at an acidic pH (that increase being in comparison to the affinity of that antibody for the same antigen at a different (e.g. neutral or alkali) pH). The pH sensitive antibody (or fragment thereof) may bind to (or have affinity/specificity for) CTLA-4 (as disclosed in WO 2019/152413, the entire contents of this disclosure being incorporated herein by reference), PD-L1 (as described in WO2017/161976: the entire contents of this disclosure being incorporated herein by reference), VISTA (as disclosed in US 202020055936 & WO2019/183040: the entire content of these disclosures being incorporated herein by reference). A fusion protein of this disclosure may comprise the anti-CD3 antibodies disclosed in WO 2020/247932A1, the anti-EPCAM antibodies disclosed in WO 2020/252095 or the pH sensitive antibodies described in WO 2018/218076.

Under the conditions present at a tumor site, certain cytokines, for example, IL-2, can become conditionally active. A fusion protein of this disclosure may therefore comprise a polypeptide domain that renders a cytokine mutein/IL-2 mutein of this disclosure conditionally inactive. A polypeptide domain that renders IL-2 conditionally inactive may be a polypeptide that prevents or reduces binding of IL-2 to its receptor. Polypeptide domains rendering interleukins conditionally inactive are known in the art as masking moieties or domains. Activation may be facilitated by steric changes of the fusion protein or by release of the polypeptides domain from the fusion protein. The masking domain may be fused to the IL-2 via a polypeptide linker. The polypeptide linker is preferably cleaved under conditions of the tumor microenvironment. Interleukin fusion proteins comprising releasable masking moiety are for example disclosed in WO 2020/069398A1 by Xilio.

A fusion protein of this disclosure (which fusion comprises a cytokine mutein or IL-2 mutein of this disclosure) may comprise a half-life extending molecule. For example, fusion of this disclosure may comprise a cytokine mutein or IL-2 mutein (as defined herein) fused or bound to a half-life extending molecule. The half-life extending molecule may comprise an immunoglobulin fragment, preferably an Fc molecule, a polypeptide binding domain which binds a blood serum protein, preferably a polypeptide binding domain which binds albumin or a polymer.

In this regard, the term “Fc molecule” may comprise a human IgGl Fc. In one teaching a useful IgGl Fc molecule may comprise one or more mutations which alter the effector function of said Fc. By way of example, a human IgGl may comprise a substitution at N297, for example a N297G substitution. In other teachings, useful human IgG Fc molecules may comprise a substitution or deletion of the C-terminal lysine.

A fusion of this disclosure may comprise a linker moiety which connects the mutein component to the other component of the fusion. A suitable linker may comprise the linker disclosed in WO2021030602A1 (the relevant contents of which are incorporated herein). In one teaching a fusion of this disclosure may comprise a cytokine/IL-2 mutein linked (via some short peptide linker) to linker connects the Fc and human IL-2 mutein portions of said protein.

A polymer (for inclusion in a fusion of this disclosure) may comprise a polyethylene glycol molecule.

This disclosure further provides the fusion proteins of this disclosure for use in methods, compositions and medicaments for the treatment and/or prevention of a range of diseases and/or conditions.

By way of example, the disclosure provides any of the disclosed fusion proteins for use in medicine. In one teaching, the disclosure provides a fusion protein comprising a cytokine mutein or an IL-2 mutein, a protein comprising SEQ ID NO: 2, 9, 10, 11, 12, 13 or a fragment of any of these, for use in medicine.

The disclosed fusion proteins may find application or use as an immunotherapy.

The disclosure provides any one of the disclosed fusion proteins for use in the treatment of an immunological condition.

The disclosure provides a fusion protein of this disclosure for use in the treatment of cancer. In one teaching, the term ‘cancer’ includes cancers, the (tumour) cells of which are characterised by the over production of lactic acid. The term ‘cancer’ may also include any cancer yielding a tumour which creates an acidic microenvironment.

Also disclosed is the use of a fusion protein of this disclosure in the manufacture of a medicament for the treatment of (i) a cancer, or (ii) an immunological condition.

The disclosure further provides a method of treating cancer, said method comprising administering a subject in need thereof a therapeutically effective amount of any of the fusion proteins disclosed herein.

A subject to be administered a fusion protein of this disclosure may include any human or animal subject suffering from an immunological condition and/or a cancer.

The subject may also be any human or animal subject predisposed and/or susceptible to an immunological condition or cancer which can be treated and/or prevented with a fusion protein of this disclosure.

It should be noted that any of the disclosed cytokine muteins, IL-2 muteins or fusion proteins may be provided in the form of a composition. Such compositions may find use as medicaments. Compositions of this disclosure may be pharmaceutical compositions comprising, for example, one or more pharmaceutically acceptable excipients.

Alternatively, compositions used as medicaments according to the present disclosure may comprise a polynucleotide, preferably an RNA most preferably an mRNA encoding any of the disclosed cytokine muteins, IL-2 muteins or fusion proteins.

The disclosed compositions comprising a protein or a polynucleotide, preferably a mRNA, may for example be administered systemically, or locally by intra- or extra-tumoral administration.

A composition comprising any of the disclosed cytokine muteins, IL-2 muteins or fusion proteins may further comprise one or more additional active or therapeutic agents. For example, the composition may comprise an anti-tumour antigen antibody, a checkpoint molecule, an antibody against a checkpoint molecule, a tumor antigen a steroid and/or a CAR T-cell.

Any of the therapeutic treatments described herein may further comprise the use of one or more additional active or therapeutic moieties, for example an anti-tumour antigen antibody, a checkpoint molecule, an antibody against a checkpoint molecule, a tumor antigen, a steroid and/or a CAR T-cell, which could be administered separately, before, during (concurrently or together with) or after, the administration of a cytokine mutein, IL-2 mutein or fusion protein of this disclosure.

In one teaching, the additional active or therapeutic moiety comprises an antibody directed against a checkpoint molecule selected from CD27, CD137, 2B4, TIGIT, CD155, CD160, ICOS, HVEM, CD40L, LIGHT, LAIR1, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDO1, ID02, TDO, KIR, LAG-3, TIM-3, or VISTA, most preferably PD-L1, PD1, or PD-L2.

For example, the disclosure provides a cytokine mutein, a IL-2 mutein and/or a fusion protein of this disclosure and one or more additional therapeutic or pharmaceutically active moieties, for use in methods, compositions and medicaments for the treatment and prevention of cancer (as defined herein) and/or an immunological conditions.

Any of the disclosed cytokine muteins, IL-2 muteins or fusion proteins may be used as adjuvants. An ‘adjuvant’ is a compound which augments, modulates or enhances a host immune response to the one or more antigens co-administrated with the adjuvant. Accordingly, the disclosed cytokine muteins, IL-2 muteins or fusion proteins may be used in combination with one or more antigens to augment, modulate or enhance a host immune response to the one or more antigens. The one or more antigens may comprise, for example microbial, bacterial and/or viral antigens.

Accordingly, the disclosure further provides a method of raising an immune response in a host to an antigen, said method composing administering the antigen and any of the disclosed cytokine muteins, IL-2 muteins or fusion proteins to the host. Without wishing to be bound by theory, the cytokine mutein, IL-2 mutein or fusion protein acts as an adjuvant to augment, modulate or enhance the immune response in the host to the antigen.

The disclosure further provides a vaccine composition comprising an antigen and any of the disclosed cytokine muteins, IL-2 muteins or fusion proteins. In a vaccine composition of this type, the cytokine mutein, IL-2 mutein or fusion protein component acts or serves as an adjuvant. In one teaching, the vaccine is a tumour vaccine.

### DETAILED DESCRIPTION

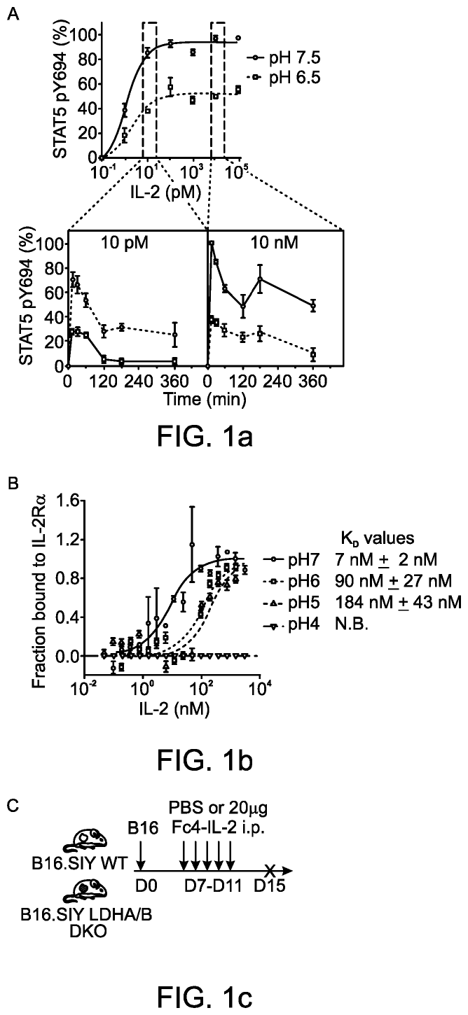

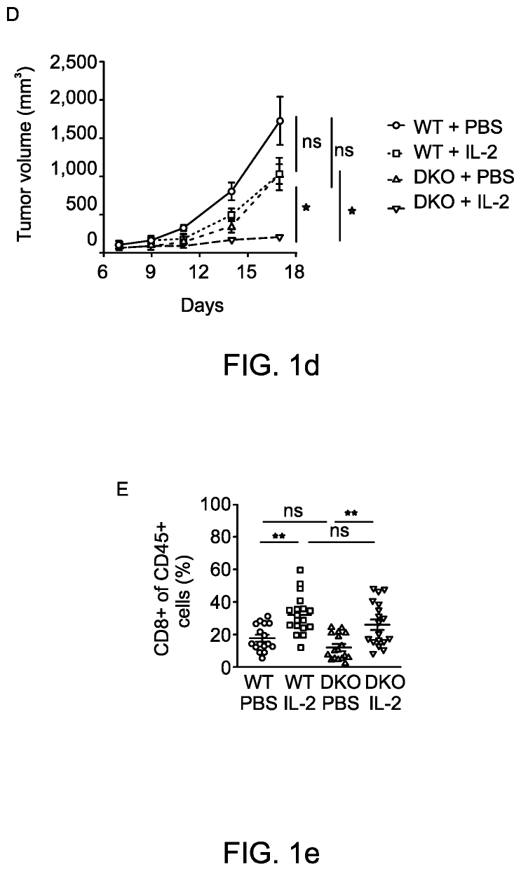

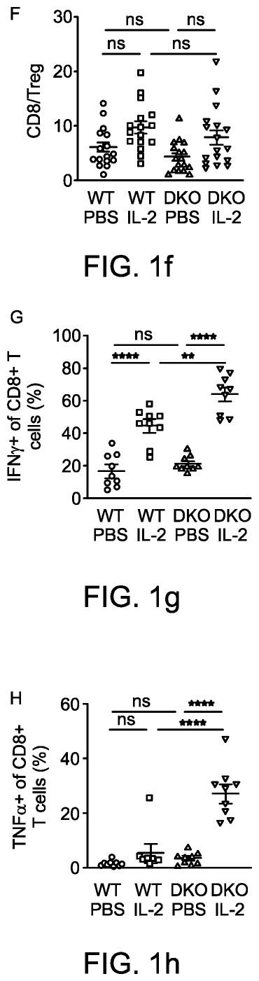

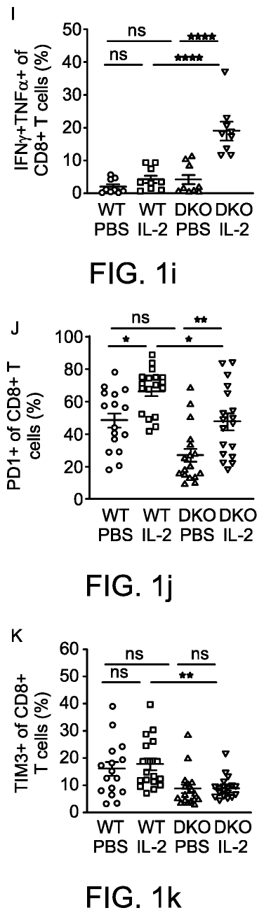

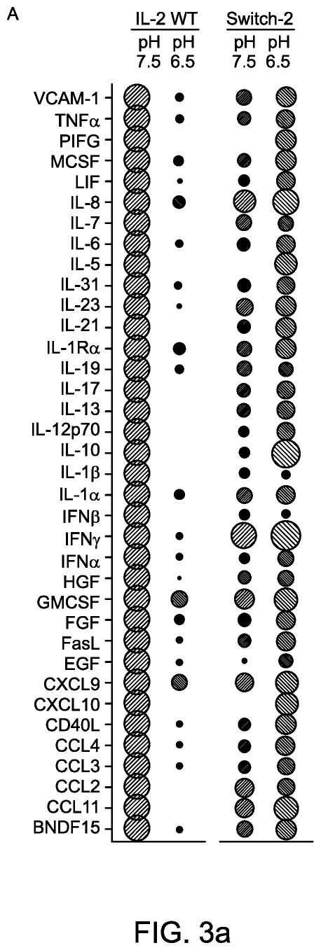

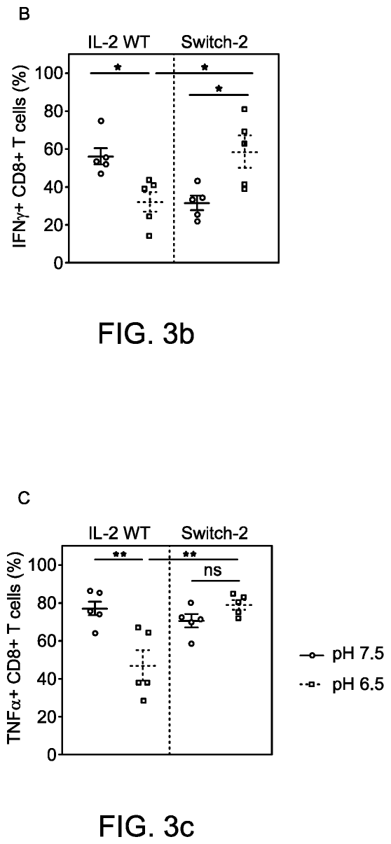

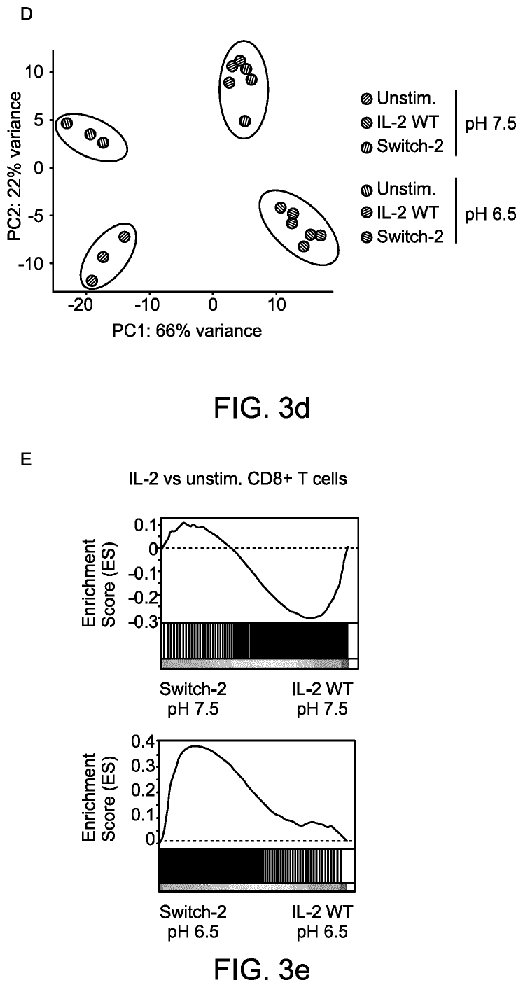

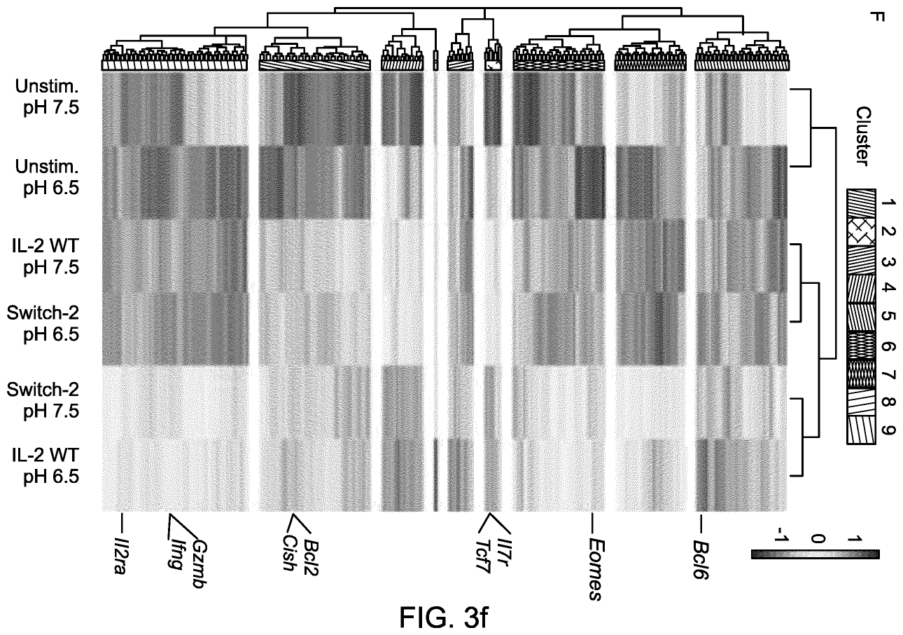

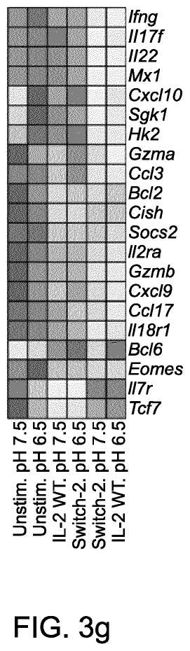

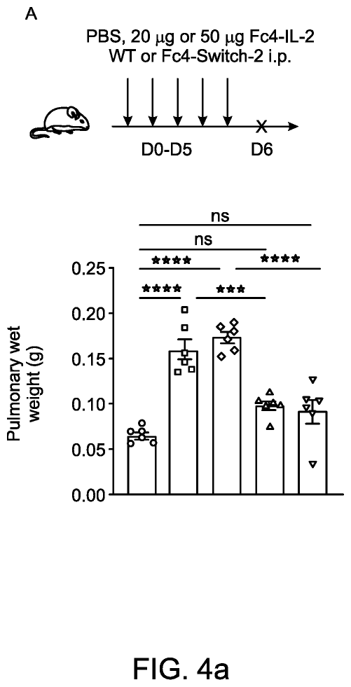

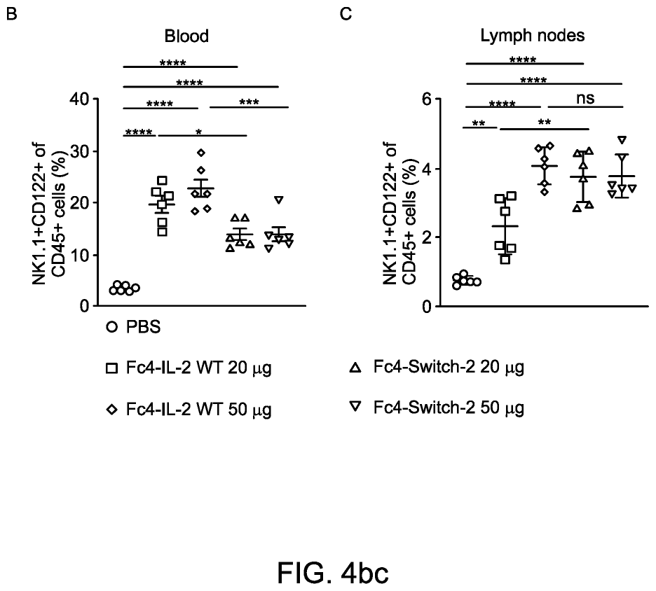

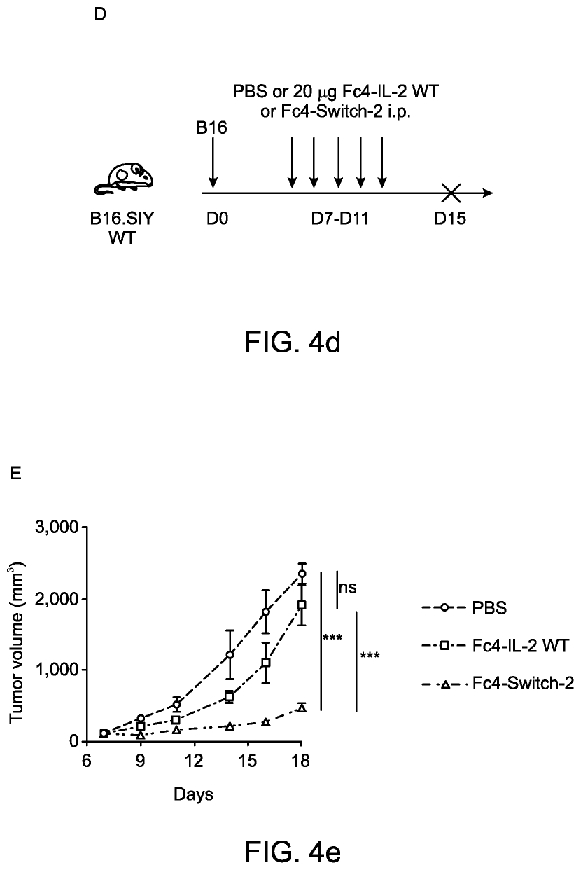

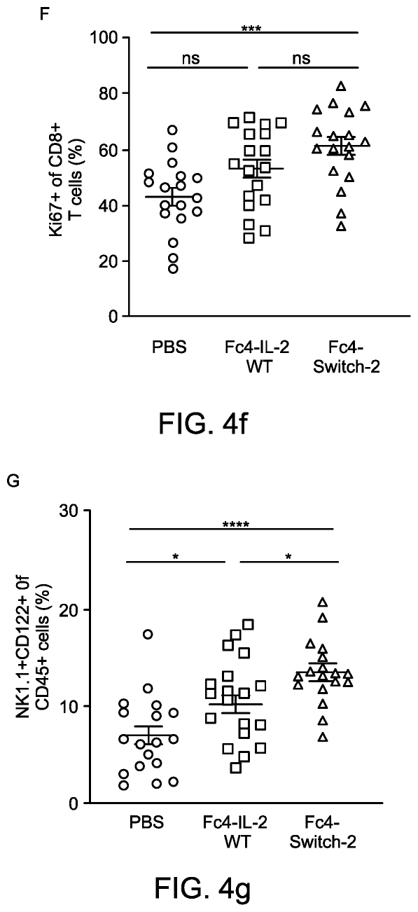

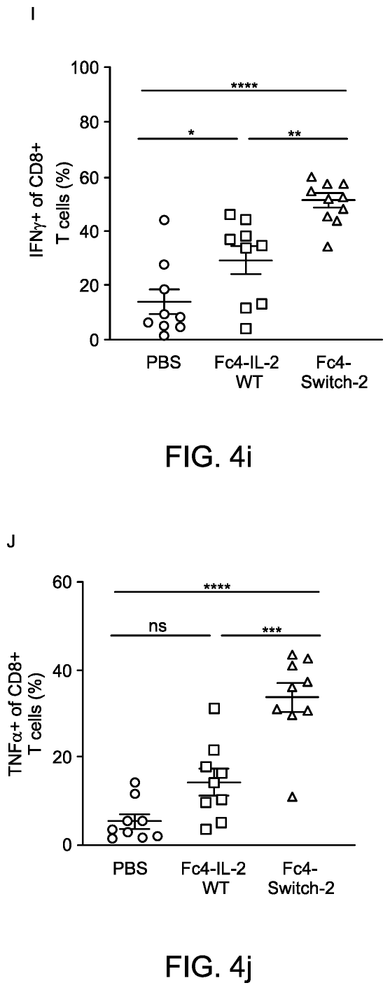

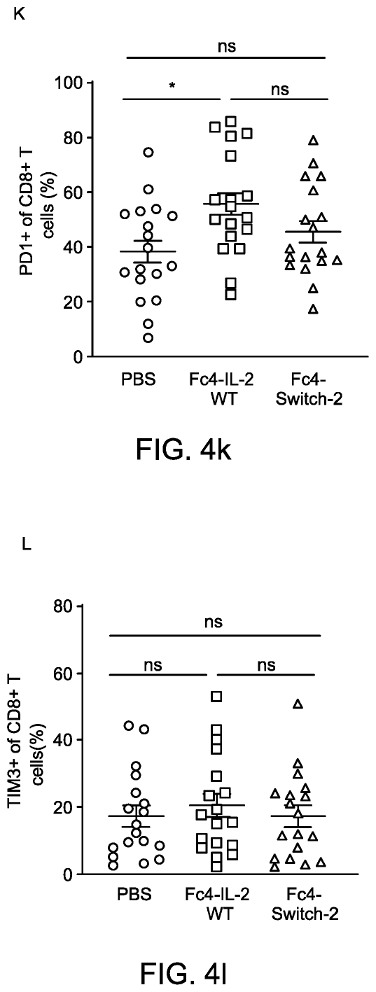

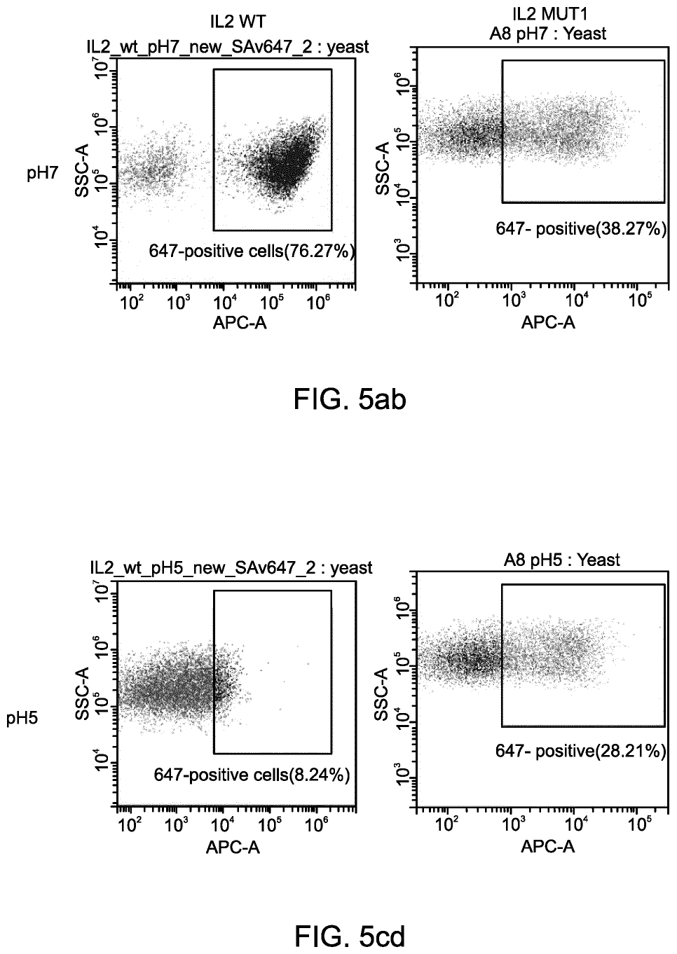

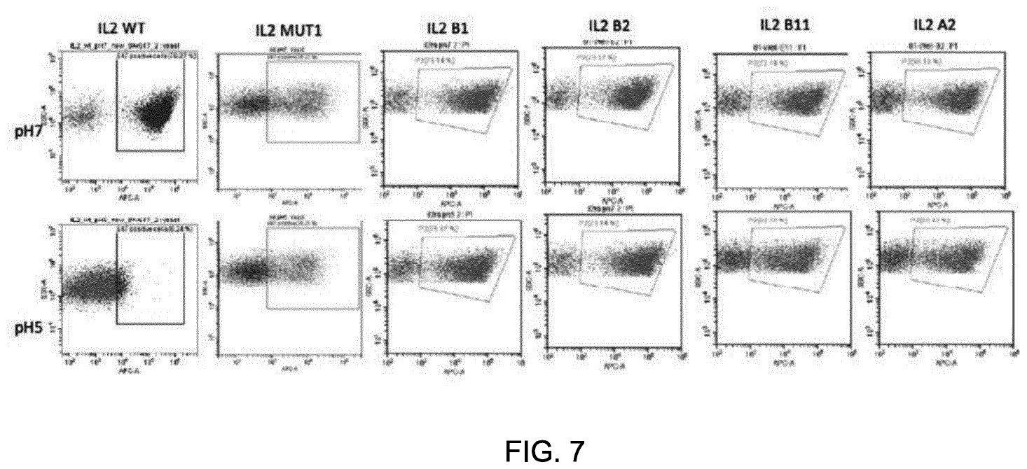

The present invention will now be described with reference to the following Figures which show:

[FIG. 1a], [FIG. 1b], [FIG. 1c], [FIG. 1d], [FIG. 1e], [FIG. 1f], [FIG. 1g], [FIG. 1h], [FIG. 1i], [FIG. 1j], [FIG. 1k]: Low pH impairs IL-2 activity. (A) Dose-response of STAT5 phosphorylation in pre-activated CD8+ T cells stimulated with IL-2 in pH 7.5 or 6.5 (top panel) and signalling kinetics at suboptimal (10 μM) and saturating dose (10 nM) (bottom panel). The graphs represent the mean±s.e.m. of three independent experiments in duplicates. (B) Microscale thermophoresis (MST) analysis of IL-2 interaction with IL-2Ra at different pH. For each condition, the Kd values are indicated. Data are mean±s.e.m. (C) Flowchart of the in vivo experiments. (D) Tumour growth of B16.SIY WT- or LDHA/B DKO-bearing mice treated with i.p. injections of 100 μl PBS or 20 μg of Fc4-IL-2 (equivalent to 7 μg of IL-2) for 5 days. Treatment was started when the tumour reached a size of 50-100 mm3. Graphs show one representative result out of 3 independent experiments (n=6 for each group). *p=0.0238 (B16.SIY WT+Fc4-IL-2 vs B16.SIY DKO+Fc4-IL-2); *p=0.0242 (B16.SIY DKO+PBS vs B16.SIY DKO+Fc4-IL-2). Data are mean±s.e.m. and significance was determined by one-way ANOVA with Tukey's correction. ns=not significant. (E-K) Analysis of tumours obtained from mice sacrificed at day 15 after tumour inoculation. (E) Percentage of CD8+ T cells. **p=0.001 (B16.SIY WT+PBS vs B16.SIY WT+Fc4-IL-2); **p=0.0018 (B16.SIY DKO+PBS vs B16.SIY DKO+Fc4-IL-2). (F) Ratio between CD8+T and Treg cells. (G-I) Cells stimulated with PMA/ionomycin were analysed for cytokine production. Percentages of IFN-γ+ (G), TNF+ (H), IFN-γ+TNF+ (D and CD8+ T cells are shown. (G) **p=0.0013; ****p<0.0001. (H, I) ****p<0.0001. (J) Percentage of PD1+CD8+ T cells. *p=0.0191 (B16.SIY WT+PBS vs B16.SIY WT+Fc4-IL-2); *p=0.0102 (B16.SIY WT+Fc4-IL-2 vs B16.SIY DKO+Fc4-IL-2); **p=0.0074. (K) Percentage of TIM3+CD8+ T cells. **p=0.0067. (E-K) Graphs show pooled results of three independent experiments. Significance was determined by one-way ANOVA with Tukey's correction. ns=not significant. Data are mean±s.e.m. and each symbol represents a single tumour (E, F, J, K) or two tumours pooled together (G-I).

[FIG. 2a], [FIG. 2b], [FIG. 2c], [FIG. 2d], [FIG. 2e], [FIG. 2f], [FIG. 2g]: Selection of pH-resistant IL-2 variant. (A) Representation of the IL-2 protein library (blue) expressed at the yeast surface and interacting with the biotinylated IL-2Rα tetramer (yellow). Amino acid that were mutated during the generation of the IL-2 library are displayed in red. (B) The mean fluorescence intensity (MFI) of the yeast displaying the IL-2 library after each round of selection at pH 5 is shown. (C) Structure of IL-2-IL-2Ra receptor complex. IL-2Rα is in yellow and IL-2 is in blue. The mutations identified in Switch-2 are highlighted in red and indicated in the right panel. (D) Dose-dependent binding at different pH of IL-2Rα serial dilutions to the surface of yeast expressing IL-2 WT or Switch-2. The graph represents the mean±s.e.m. of two independent experiments. (E) Quantification of the IL-2/IL-2Rα interaction at the plasma membrane of live cell by dual colour TIRF microscopy with labelled IL-2Rα and IL-2 (left panel) and graph showing the IL-2 binding normalised to the IL-2Rα cell surface expression. Data are mean±s.e.m. with each data point representing the result from a single cell. Significance was calculated by Kolmogorov-Smirnov test. ****p<0.0001. (F, G) Dose-response curve of phospho-STAT5 (pSTAT5) induced by IL-2 WT and Switch-2 at pH 7.5 and 6.5 in freshly isolated (F), and pre-activated CD8 T cells (G). The graphs represent the mean±s.e.m. of three independent experiments in duplicates.

[FIG. 3a], [FIG. 3b], [FIG. 3c]: Functional in vitro activity of IL-2 C1 in acidic pH. (A-C) Analysis of cytokine expressed by pre-activated CD8+ T cells after 3 days of culture at pH 7.5 or 6.5 in the presence of 10 nM IL-2 WT or Switch-2. Cells were stimulated with PMA/ionomycin. (A) Supernatant of stimulated cells was analysed by Luminex assay. The bubbles represent the amount of the released cytokines that has been normalised to control condition (IL-2 WT pH 7.5=100). Data are mean of three different donors. (B, C) Graphs represent the percentage of IFN-γ+ (B) and TNF+ (C) cells. Data are mean±s.e.m. and each symbol represents a single donor. (B) *p=0.0414 (IL-2 WT pH 7.5 vs IL-2 WT pH 6.5); *p=0.0233 (IL-2 WT pH 6.5 vs Switch-2 pH 6.5); *p=0.0195 (Switch-2 pH 7.5 vs Switch-2 pH 6.5) by one-way ANOVA with Tukey's correction. (C) **p=0.0027 (IL-2 WT pH 7.5 vs IL-2 WT pH 6.5); **p=0.0015 (IL-2 WT pH 6.5 vs Switch-2 pH 6.5) by one-way ANOVA with Tukey's correction. (D) Principal component analysis (PCA) of RNA-seq data. Pre-activated CD8+ T cells from three different donors were stimulated for 4 h after resting O/N. (E) GSEA analysis of Switch-2—versus IL-2 WT-stimulated CD8+ T cells at pH 7.5 (top panel) and at pH 6.5 (bottom panel). (F, G) Heatmap of the 476 top variable and significant genes (F) and of a set of T cell-specific genes (G). Gene expression is represented as z-score.

[FIG. 4a], [FIG. 4bc], [FIG. 4d], [FIG. 4e], [FIG. 4f], [FIG. 4g], [FIG. 4i], [FIG. 4j]: Switch-2 improves survival and stimulates anti-tumour immune response. (A) Pulmonary oedema (pulmonary wet weight) was evaluated in mice treated with either PBS, 20 μg or 50 μg of Fc4-IL-2 WT or Switch-2 by weighing lungs before and after drying. Data are mean±s.e.m. of two independent experiments and each symbol represents a mouse. ***p=0.0002 (Fc4-IL-2 WT 20 μg vs Fc4-Switch-2 20 μg); ****p<0.0001. (B, C) Percentage of NK in the blood (B) and lymph nodes (C) of mice treated with either PBS, 20 μg or 50 μg of Fc4-IL-2 WT or Switch-2. (B) *p=0.0338 (Fc4-IL-2 WT 20 μg vs Fc4-Switch-2 20 μg); ***p=0.0006 (Fc4-IL-2 WT 50 μg vs Fc4-Switch-2 50 μg); ****p<0.0001. (C) **p=0.0031 (Fc4-IL-2 WT 20 μg vs Fc4-Switch-2 20 μg); **p=0.0014 (PBS vs Fc4-IL-2 WT 20 μg); ****p<0.0001. (D) Flowchart of the in vivo experiments. (E) Tumour growth of B16.SIY WT-bearing mice treated with i.p. injections of 100 μl PBS or 20 μg of Fc4-IL-2 WT or Fc4-Switch-2 for 5 days. Treatment was started when the tumour reached a size of 50-100 mm3 (n=6 for each group). Graphs show representative result of three independent experiments. ***p=0.0005 (PBS vs Fc4-Switch-2), ***p=0.0007 (Fc4-IL-2 WT vs Fc4-Switch-2). Data are mean±standard deviation and significance was determined by one-way ANOVA with Tukey's correction. ns=not significant. (F-L) Analysis of tumours obtained from mice sacrificed at day 15 after tumour inoculation. (F) Percentage of Ki67+CD8+ T cells. ***p=0.0006. (G) Percentage of NK1.1+CD122+ cells. *p=0.0359 (PBS vs Fc4-IL-2 WT); *p=0.0334 (Fc4-IL-2 WT vs Fc4-Switch-2); ****p<0.0001 (I, J). Percentages of CD8+IFN-γ+ (i) and CD8+TNF+ (J) T cells after stimulation with PMA/ionomycin. (I) *p=0.0⁴⁶⁵; **p=0.00³⁵; ****p<0.0001. (J) ***p=0.0001; ****p<0.0001. (K) Percentage of PD1+CD8+ T cells. *p=0.015⁸. (L) Percentage of TIM3+CD8+ T cells. (A-C, E-L) Data are mean±standard deviation and each symbol represents a single mouse (A-C, F, G, K, L) or two mouse pooled together (I, J) of two (A-C) and three (F-L) independent experiments. Significance was determined by one-way ANOVA with Tukey's correction. ns=not significant.

[FIG. 5ab], [FIG. 5cd]: Analysis of pH dependent binding of yeasts displaying IL-2 WT at pH 7 (FIG. 5 a) and pH 5 (FIG. 5 c) or IL-2 MUT1 at pH 7 (FIG. 5 b) and pH 5 (FIG. 5 d) by flow cytometry.

[FIG. 6]: Alignment of SEQ IDs 1, 2 to 8: SEQ ID NO: 1 of mature human IL-2, Accession no. P60568; SEQ ID NO 3 of mature mouse IL-2, Accession no. P04351; SEQ ID NO 4 of mature rat IL-2, Accession no. P17108; SEQ ID NO 5 of mature pig IL-2, Accession no. P26891; SEQ ID NO 6 of mature fox IL-2, Accession no. Q25BC3; SEQ ID NO 7 of mature dog IL-2, Accession no. NP_001003305; and SEQ ID NO 8 of mature macaca IL-2, Accession no. P68291. Residues 37, 38, 41, 42, 43, 64 in SEQ ID NO:1 and respective residues in other SEQ ID NOs are marked in bold.

[FIG. 7]: Description of additional IL-2 pH resistant mutants. Dot plots of IL-2 mutants binding to IL-2Rα at pH5 and pH 7.

### MATERIALS AND METHODS

### Cell Culture and Media

B16.SIY WT and B16.SIY LDHA/B DKO (kindly provided by Marina Kreutz, University of Regensburg) were cultured in RPMI 1640 with GlutaMAX supplemented with 10% foetal bovine serum (FBS) and penicillin/streptomycin. HeLa cells stably transfected with SNAPf-IL-2Rα were cultivated at 37° C. and 5% C02 in MEM medium supplemented with Earle's balanced salts, glutamine, 10% FBS, non-essential amino acids, and HEPES buffer without addition of antibiotics. For baculovirus preparation and protein production, Spodoptera frugiperda (Sf9) and Trichoplusia ni (High Five) cells were cultured in SF900 III SFM media (Invitrogen; 12658027) and in Insect Xpress media (Lonza; BELN12-730Q), respectively. Human T cells were cultured in RPMI 1640 with GlutaMAX (Gibco, 61870036) supplemented with 10% FBS, minimum non-essential amino acids, 1 mM sodium pyruvate, and penicillin/streptomycin. When the pH of the media was adjusted to conduct short or long term experiments, HCl was used to acidify the media and 20 mM HEPES pH 6.5 was added to maintain stable the pH at 6.5. An equivalent amount of HEPES pH 7.5 was added to the media at pH 7.5. In the case of murine T cells, the media was further supplemented with 50 μM β-mercaptoethanol.

### Protein Production

Human IL-2 wild-type (WT; residues 1-133) and Switch-2 were cloned into the pFB-CT10HF vector in frame with the N-terminal gp67 and the C-terminal histidine tag; human IL-2Rα ectodomain (residues 1-217) was cloned in the same vector with a C-terminal biotin acceptor peptide (BAP)-LNDIFEAQKIEWHW followed by a histidine tag; for in vivo experiments, the Fc portion of human IgG4 was cloned at the N-terminal of IL-2 WT and Switch-2. Proteins were produced using the baculovirus expression system. Briefly, vectors were recombined in DH10Bac bacteria (Gibco) and the generated bacmid were used to generate the baculovirus. Baculovirus was produced and amplified in Spodoptera frugiperda (Sf9) cells and used to infect Trichoplusia ni (High Five) cells for protein expression. Two days after infection, His-Pur Ni-NTA resin (Invitrogen; 88222) was used to capture the proteins released in the cell culture supernatant. Proteins were purified by size exclusion on a Superdex 75 Increase column (GE Healthcare; 29-1487-21). Proteins were conserved in 10 mM HEPES (pH 7.2) and 150 mM NaCl (HBS buffer). In the case of IL-2Rα, the protein was reduced with 10 mM cysteine, alkylated with 20 mM iodoacetamide14, and biotinylated with BirA ligase in the presence of 100 μM biotin. For microscopy experiments, IL-2 WT and Switch-2 were cloned into pMAL vector in frame with N-terminal Mannose Binding Protein (MBP) and YbbR tag (DSLEFIASKLA peptide), and a C-terminal histidine tag. BL21 Escherichia coli cells were used to express the protein upon O/N induction with 1 mM IPTG at 20° C. The periplasmic fraction was isolated by osmotic shock and recombinant proteins were captured by His-Pur Ni-NTA resin. Proteins were purified by size exclusion on a Superdex 75 Increase column.

### Microscale Thermophoresis (MST)

MST was conducted using a NT.115 Pico MST instrument (Nano Temper Technologies GmbH) equipped with red and blue filter sets. IL2 WT and Switch-2 were diluted to 200 nM in PBS buffer with 0.05% Tween (PBS-T) and labelled with Monolith His-Tag Labeling Kit RED-tris-NTA (Nano Temper; MO-L018). The RED-tris-NTA dye was diluted in PBS-T to 100 nM. The mix was incubated at room temperature (RT) in the dark for 30 min. IL-2Rα ectodomain (25 μM) was diluted with a serial 1:1 ratio of 16 gradients. Then the labelled protein and IL-2Rα ectodomain were mix with 1:1 ratio and incubated at RT in the dark for 15 min. Capillaries are then filled individually and loaded into instrument. Data were acquired using medium MST power and 20% LED. Data were analysed using MO Control Software (Nano Temper). MST figures were rendered using MO Affinity Analysis (Nano Temper) and GraphPad Prism 7.

### Human T Cell Isolation and Culture

Peripheral Blood Mononuclear Cells (PBMCs) of healthy donors were isolated from buffy coats (Etablissement Frangais du Sang) by density gradient centrifugation using Pancoll human (Pan Biotech, P04-60500). 200×106 PBMCs were stained with 15 μl of anti-CD8 FITC antibody (Clone HIT8a; Biolegend, 300906) for 15 min at 4° C., washed and incubated with 70 μl anti-FITC microbeads (Miltenyi, 130-048-701). CD8+ T cells were isolated by magnetic separation using LS columns (Miltenyi, 130-042-401) and activated for 3 days in complete media using coated anti-CD3 antibody (Clone OKT3; Biolegend, 317326) and 2 μg/ml soluble anti-CD28 antibody (Clone CD28.2; Biolegend, 302934). Activation was always carried on at neutral pH 7.5, except when specifically indicated. For proliferation assay, CD8+ T cells were labelled with CellTrace Violet (Thermo Scientific, C34557) prior to T cell activation following the manufacturer protocol. For mRNA purification, activated CD8+ T cells were rested O/N, transferred in complete media pH 7.5 or 6.5 and stimulated for 4 h with 10 nM IL-2 WT or Switch-2. In the case of CD8+ T cells used for proteomic analysis, activated cells were cultured for 48 h in media at pH 7.5 or 6.5 in the presence of 10 nM IL-2 WT or Switch-2, washed twice with PBS and the dry cell pellet was frozen. Activated CD8+ T cell used for analysing cytokine expression and for secretome analysis were cultured for 3 days in media at pH 7.5 or 6.5 in the presence of 10 nM IL-2 WT or Switch-2 and subsequently stimulated for 4 h. Cell stimulation cocktail containing transport inhibitors (eBioscience; 00-4975-93) was used for cytokine expression analysis by flow cytometry. Supernatant for Luminex analysis were collected upon stimulation with cell stimulation cocktail (eBioscience; 00-4970-93). CD4+ cells were isolated using 40 μl of anti-CD4 FITC antibody (Clone A161A1; Biolegend; 357406) following the same protocol of CD8+ T cell isolation.

### Signalling Experiments

For signalling experiments, activated CD8+ T cells rested O/N and subsequently cells were stimulated for 15 min with the indicated amount of IL-2 WT or Switch-2 in media at pH 7.5 or 6.5. In the case of time-course experiments cells were stimulated for 6 h, 3h, 2 h, 1 h, 30 min, 15 min with 10 nM or 10 μM IL-2. IL-2 signalling in Treg cells was evaluated after 15 min stimulation of freshly isolated total CD4 cells.

### Flow Cytometry Analysis

Human CD8 cells were incubated with Zombie aqua Fixable viability kit (Biolegend; 423101) for 20 min at 4° C. and then stained for surface markers 30 min at 4° C. in MACS buffer (Miltenyi; 130-091-221) using anti-human CD8 FITC, anti-human CD3 BV711 (clone UCHT1; Biolegend; 300463) anti-human CD25 APC (clone M-A251; Biolegend, 356110), anti-human CD122 PE-Cy7 (clone TU27; Biolegend. 339013), anti-human CD132 PE (clone TUGh4; Biolegend; 338605), anti-human CD69 BV650 (clone FN50; Biolegend; 310933). For the analysis of cytokine expression, cells stained for surface markers were subsequently fixed and permeabilized using BD Cytofix/Cytoperm kit (BD Biosciences; 554714). Anti-human IL-2 BV421 (clone MQ1-17H12; Biolegend, 500328), anti-human TNFα PE/Dazzle 594 (clone Mab11; Biolegend, 502946), and anti-human IFNγ APC (clone B27; Biolegend, 506510) were used. All the antibodies were used at 1:100. For dose-response and kinetic experiments, stimulated cells were immediately fixed with 2% PFA for 15 min at RT. Cells were subsequently washed with PBS and permeabilized with ice-cold methanol for 30 min on ice and fluorescently barcoded as previously described15. In brief, individual wells were stained with a combination of different concentrations of PacificBlue (Thermo Scientific; 10163) and DyLight800 NHS-dyes (Thermo Scientific; 46421). 16 barcoded samples were pooled together and stained with 1:100 anti-STAT5 PE (clone 47/Stat5; BD Biosciences; 612567), 1:100 anti-ERK1/2 AF647 (clone 4B11B69; Biolegend, 677504), 1:50 anti-Akt AF647 (clone 193H2; Cell Signaling Technologies, 2337S), and 1:100 anti-S6R PE (clone D57.2.2E; Cell Signaling Technologies; 5316S) in MACS buffer for 1 h at RT. In the case of signalling experiments on Treg cells, samples were washed and stained with 1:10 anti-human FoxP3 AF647 (clone 259D/C7; BD Biosciences; 560045) using the FoxP3/transcription factor staining buffer set (eBioscience; 00-5523-00). Single cell suspension of murine spleen and lymph nodes was obtained by mechanical disruption. Tumours were digested with collagenase (Sigma, C6885) and DNase I (StemCell, 07470). After treatment with TruStain FcX (anti-mouse CD16/32) Antibody (Biolegend; 101320), samples were stained following the same procedure described before. The following antibodies were used: anti-mouse CD3 PerCP-Cy5.5 (clone 17A2; Biolegend; 100218), anti-mouse CD4 BV605 (clone RM4-5; Biolegend; 100548), anti-mouse CD4 AF700 (clone GK1.5; Biolegend; 100430), anti-mouse CD8 AF488 (clone 53-6.7; Biolegend 100723), anti-mouse-CD45 BV711 (clone 30-F11; Biolegend; 103147), anti-mouse CD122 PE/Dazzle 594 (clone TM-01; Biolegend; 123217), anti-mouse PD-1 BV785 (clone 29F-1A12; Biolegend; 135225), anti-mouse TIM3 BV421 (clone RMT3-23; Biolegend; 119723), anti-mouse FoxP3 PE (clone FJK-16s; eBioscience; 12-5773-82), anti-mouse Ki67 PE-Cy5 (clone SolA15; eBioscience; 15-5698-82), anti-mouse NK1.1 BV605 (clone PK136; Biolegend; 108739), anti-mouse TNFα BV605 (clone MP6-XT22; Biolegend; 506329), anti-mouse IFNγ APC (clone XMG1.2; Biolegend; 505809). Flow cytometry was performed using LSR Fortessa X20 (BD) instrument and data were analysed with FlowJo software (TreeStar Inc, version 10).

### Animal Models

6-weeks old female C57Bl/6JRj mice (Janvier) were subcutaneously injected in the right flank with 30.000 B16.SIY WT or B16.SIY LDHA/B DKO in PBS and Matrigel (1:1) (Corning; 356232). 20 μg of Fc4-IL-2 WT or Switch-2 were administered intraperitoneally (i.p). from day 7 to day 11. Tumour was measured using a caliper and tumour volume was calculated using the formula length×width2/2. For the analysis of TILs, mice were sacrificed at day 15 after tumour injection. For toxicity test 20 or 50 μg of Fc4-IL-2 WT or Switch-2 were given for 5 consecutive days by i.p. and mice were sacrificed the day after the last injection. Pulmonary oedema (pulmonary wet weight was evaluated by measuring the wet weight after lung collection and subtracting the dry weight after the lungs were desiccated O/N at 80° C.

### Generation and Selection of IL-2 Library

Adapting a previously described protocol for yeast display 16, we cloned IL-2 cDNA in pCT302 vector for the expression in yeast. The IL-2 library was generated assembling 8 overlapping primers, among which two of them containing the homology regions necessary for the combination with the pCT302 vector (Table 1). Three of the primers had NDT codons (encoding for G, V, L, I C, S, R, H, D, N, F, Y amino acids) used to randomly mutate T37, R38, T41, F42, F43, E60, E61, E63, L66, E68, V69, D109, and E110 residues. The PCR product was further amplified using Lib Fw and Lib Rv primers (Table 1), at a final concentration of 10 μM, to obtain at least 25 pig of DNA. S. cerevisiae strain EBY100 was transformed by electroporation with 25 μg of insert DNA and 5 μg of the linearized and purified plasmid. Transfected yeasts were grown in SDCAA media for 1 day at 30° C. and in SGCAA for 2 days at 20° C. at each round of selection. The library, with a size of 5×107,

was screened by magnetic-activated cell sorting (MACS) using LS column (Miltenyi; 130-042-401): the first round of selection was carried on with 1010 cells and the subsequent ones with 108 cells to ensure at least 10-fold coverage for each round. Biotinylated IL-2Rα ectodomain was used at different concentrations to select pH-resistant IL-2 variants: more in detail, the first two rounds were performed using IL-2Rα tetramer at 100 nM in pH 5, the third and fourth round with 1 μM and 100 nM IL-2Rα monomer, respectively. IL-2Rα tetramers were generated by incubating IL-2Rα and Streptavidin (SA)-Alexa 647 at a ratio of 4:1.

TABLE 4

| Primer | |
|---|---|
| name | 5′-3′ sequence |
| Lib | AGCGGTGGGGGCGGTTCTCTG |
| Fw | |
| Lib | TCGAGCAAGTCTTCTTCGGAG |
| Rv | |
| IL-2 | AGCGGTGGGGGCGGTTCTCTGGAAGTTCTGTTCCAGGGTCCGAGC |
| Amp | GGC |
| Fw | GGATCCGCACCTACTTCAAGTTCTACAAAGAA |
| IL2 | GCACCTACTTCAAGTTCTACAAAGAAAACACAGCTACAACTGGA |
| Lib | GCATTTA |
| Fw 1 | CTGCTGGATTTACAGATGATTTTGAATGGAATTAAT |
| IL2 | GATGTTTCAGTTCTGTGGCCTTCTTGGGCATGTAAAAahnahnahnGA |
| Lib | GCAT |
| Rv 1 | ahnahnGAGTTTGGGATTCTTGTAATTATTAATTCCATTCAAAATCA |
| | T |
| IL2 | CCACAGAACTGAAACATCTTCAGTGTCTAndtndtndtCTCAAACCTnd |
| Lib | tGAGnd |
| Fw 2 | tndtCTAAATTTAGCTCAAAGCAAAAACTTTCACTTAAGACCCAG |
| IL2 | CATGAATGTTGTTTCAGATCCCTTTAGTTCCAGAACTATTACGTTG |
| Lib | ATATTG |
| Rv 2 | CTGATTAAGTCCCTGGGTCTTAAGTGAAAGTTT |
| IL2 | GGGATCTGAAACAACATTCATGTGTGAATATGCTndtndtACAGCAA |
| Lib | CCATTG |
| Fw 3 | TAGAATTTCTGAACA |
| IL2 | AGTCAGTGTTGAGATGATGCTTTGACAAAAGGTAATCCATCTGTT |
| Lib | CAGAAA |
| Rv 3 | TTCTACAATGG |
| IL2 | TCGAGCAAGTCTTCTTCGGAGATAAGCTTTTGTTCGCCACCAGAA |
| Amp | GCGGC |
| Rv | CGCAGTCAGTGTTGAGATGATGCT |

### Nano Differential Scanning Fluorimetry (NanoDSF)

IL2 WT and Switch-2 (10 piM) were analysed using Tycho NT.6 (NanoTemper, Munich, Germany) by applying a standard capillary (10 μl) for each HBS buffer condition (pH 7 to 4). Thermal unfolding profiles were recorded within a temperature gradient between 35° C. and 95° C. Inflection temperatures (Ti) values were determined automatically using the integrated software.

### Single-Molecule Fluorescence Imaging

For microscopy experiments, HeLa cells stably transfected with SNAPf-IL-2Rα were transferred onto 25 mm glass coverslips coated with a poly-L-lysine-graft-(polyethylene glycol) copolymer functionalized with RGD to minimize non-specific binding 17. Single-molecule imaging experiments were conducted by total internal reflection fluorescence (TIRF) microscopy with an inverted microscope (Olympus IX71) equipped with a triple-line total internal reflection (TIR) illumination condenser (Olympus) and a back-7 illuminated electron multiplied (EM) CCD camera (iXon DU897D, Andor Technology) as previously described in more detail 18,19. A 150× magnification objective with a numerical aperture of 1.45 (UAPO 150×/1.45 TIRFM, Olympus) was used for TIR illumination of the sample. All experiments were carried out at RT in medium without phenol red, supplemented with an oxygen scavenger and a redox-active photoprotectant to minimize photobleaching 20.

For stoichiometric cell surface labelling of SNAPf-tagged IL-2Rα, cells were incubated with 80 nM of a premixed BG-dye solution (95% BG-488 and 5% BG-Dy547) at 37° C. for 15 min and washed 5 times with pre-warmed PBS to remove unreacted dyes. Dy547P1/Dy647P1 conjugated ybbR-IL-2 WT and Switch-2 at a concentration of 1 nM were added 5 min prior to imaging experiments. For single molecule experiments, orange (Dy547/Dy547P1) and red (DY647P1) emitting fluorophores were simultaneously excited by illumination with a 561 nm fiber laser (2RU-VFL-P-500-560-B1R, MPB Communications) and a 642 nm fiber laser (2RU-VFL-P-500-642-B1R, MPB Communications). Fluorescence was filtered by a penta-band polychroic mirror (zt405/488/561/640/730rpc, Semrock) and excitation light was blocked by a penta-band bandpass emission filter (BrightLine HC 440/521/607/694/809, Semrock). For simultaneous acquisition of both channels with a single back-illuminated EMCCD camera (iXon Ultra 897, Andor Technologies), a four-color image splitter (QuadView, QV2, Photometrics) was used, which was equipped with three dichroic beamsplitters at 565 nm, 630 nm, and 735 nm (480dcxr, 565dcxr, 640dcxr, Chroma) and four single-band bandpass emission filters (BrightLine HC 438/24, BrightLine HC 520/35, BrightLine HC 600/37, BrightLine HC 685/40, Chroma). Image stacks of 150 frames were recorded for each cell at a time resolution of 32 ms/frame.

Single-molecule localization was carried out using the multiple-target tracing (MTT) algorithm 21. For ratiometric ligand binding quantification, localizations of 30 frames of Dy647P1 (IL-2 WT or Switch-2) were either normalized to Dy547 (IL-2Rα) or Dy547P1 (IL-2 WT) localizations, respectively.

### Single-Molecule Fluorescence Imaging

For microscopy experiments, HeLa cells stably transfected with SNAPf-IL-2Rα were transferred onto 25 mm glass coverslips coated with a poly-L-lysine-graft-(polyethylene glycol) copolymer functionalized with RGD to minimize non-specific binding17. Single-molecule imaging experiments were conducted by total internal reflection fluorescence (TIRF) microscopy with an inverted microscope (Olympus IX71) equipped with a triple-line total internal reflection (TIR) illumination condenser (Olympus) and a back-7 illuminated electron multiplied (EM) CCD camera (iXon DU897D, Andor Technology) as previously described in more detail 18,19. A 150× magnification objective with a numerical aperture of 1.45 (UAPO 150×/1.45 TIRFM, Olympus) was used for TIR illumination of the sample. All experiments were carried out at RT in medium without phenol red, supplemented with an oxygen scavenger and a redox-active photoprotectant to minimize photobleaching 20.

For stoichiometric cell surface labelling of SNAPf-tagged IL-2Rα, cells were incubated with 80 nM of a premixed BG-dye solution (95% BG-488 and 5% BG-Dy547) at 37° C. for 15 min and washed 5 times with pre-warmed PBS to remove unreacted dyes. Dy547P1/Dy647P1 conjugated ybbR-IL-2 WT and Switch-2 at a concentration of 1 nM were added 5 min prior to imaging experiments. For single molecule experiments, orange (Dy547/Dy547P1) and red (DY647P1) emitting fluorophores were simultaneously excited by illumination with a 561 nm fiber laser (2RU-VFL-P-500-560-B1R, MPB Communications) and a 642 nm fiber laser (2RU-VFL-P-500-642-B1R, MPB Communications). Fluorescence was filtered by a penta-band polychroic mirror (zt405/488/561/640/730rpc, Semrock) and excitation light was blocked by a penta-band bandpass emission filter (BrightLine HC 440/521/607/694/809, Semrock). For simultaneous acquisition of both channels with a single back-illuminated EMCCD camera (iXon Ultra 897, Andor Technologies), a four-color image splitter (QuadView, QV2, Photometrics) was used, which was equipped with three dichroic beamsplitters at 565 nm, 630 nm, and 735 nm (480dcxr, 565dcxr, 640dcxr, Chroma) and four single-band bandpass emission filters (BrightLine HC 438/24, BrightLine HC 520/35, BrightLine HC 600/37, BrightLine HC 685/40, Chroma). Image stacks of 150 frames were recorded for each cell at a time resolution of 32 ms/frame. Single-molecule localization was carried out using the multiple-target tracing (MTT) algorithm 21. For ratiometric ligand binding quantification, localizations of 30 frames of Dy647P1 (IL-2 WT or Switch-2) were either normalized to Dy547 (IL-2Rα) or Dy547P1 (IL-2 WT) localizations, respectively.

### Luminex Analysis

Cell supernatants were measured on a custom 36-multiplex R&D systems Luminex panel (R&D Systems) in the Immunoassay Biomarker Core Laboratory, University of Dundee. The samples were diluted two-fold as instructed in the assay instructions. A Bio-Plex Pro wash station was used to perform the wash steps. The multiplex assay plate was measured on a Bio-plex 200 analyser using Bio-plex Manager software v6.1.36 different analyte-specific antibodies are pre-coated onto microparticles.

Standards, samples, and a cocktail of all the microparticles were added to each well. The plate was covered with a foil plate sealer and left to incubate, shaking at 800±50 rpm, for 2 h at RT. During this stage immobilised antibodies bind the analytes of interest. Using the Bio-Plex Pro wash station, the plate was washed three times as according to assay instructions.

Diluted biotinylated antibody cocktail specific to the analytes of interest was added to each well. The plate was covered with a foil plate sealer and left to incubate, shaking at 800±50 rpm, for 1 h at RT.

After this the plate was washed as before. Diluted streptavidin-phycoerythrin conjugate (Streptavidin-PE) was then added to each well. The plate was covered with a foil plate sealer and left to incubate, shaking at 800±50 rpm, for 30 min at RT. After this the plate was washed as before. The microparticles were resuspended in wash buffer. The plate was placed on a plate shaker for 2 min set at 800±50 rpm. The plate was read immediately using a Bio-plex 200 analyser. The mean blank Median Fluorescence Intensity (MFI) was subtracted from the mean duplicate MFI readings for each of the standards and samples. A five-parameter logistic (5-PL) curve-fit standard curve was generated for each analyte using the Bio-plex Manager v6.1 software. The software also calculated the results considering the sample dilution.

### RNAseq Analysis

RNA of human CD8+ T cells was purified using Quick-RNA Microprep kit (Zymo Research; R1051). Library preparation and sequencing were performed by Novogene.

### Murine CD8+ T Cell Isolation and Activation

Murine CD8+ T cells were isolated from mouse spleen using MagniSort mouse CD8 T cell

enrichment kit (eBioscience; 8804-6822-74). Cells were activated for 3 days in complete media using coated anti-CD3 antibody (Clone 145-2C11; Biolegend; 100340) and 2 μg/ml soluble anti-CD28 antibody (Clone 37.51; Biolegend; 102116).

### Statistical Analysis

Multiple group comparisons were performed using one-way ANOVA with Tukey's correction. Survival curves are represented as Kaplan-Meier curves and statistical significance was determined by Log-rank test. All the analysis were performed using Prism 9 software (GraphPad).

### Results

### Acidic pH in the TME Impairs IL-2-Immuno Therapy

Early studies suggested that binding of IL-2 to its receptors is a pH sensitive process7. Thus, we sought to determine whether acidic pHs, as the one found in the TME, affected IL-2 signalling and activities. IL-2 triggered significantly lower levels of STAT5 phosphorylation in pre-activated CD8+ T cells cultured at pH 6.5 than in cells cultured at pH 7.5 (FIG. 1a and Extended Data FIG. 1a). Similar results were found when the media was acidified using lactic acid (Extended Data FIG. 1b). By replacing HCl or lactic acid with NaCl, we found that the effect of acidic pH on IL-2 signalling was independent of tonicity as equimolar concentration of NaCl failed to replicate the pH effect on signalling. However, in IL-2Rα negative cells that weakly respond to IL-2, IL-2 activated STAT5 to the same extent at pHs 6.5 and 7.5, suggesting that binding of IL-2 to IL-2Rα is pH sensitive (Extended Data FIG. 1c, d). Indeed, yeast-displayed IL-2 bound IL-2Rα consistently worse at pHs 6 and 5 than 7 (FIG. 1b). We confirmed these results by measuring the dissociation constant (Kd) of the recombinant proteins (Extended Data FIG. 1e). Next, we investigated whether the acidic pH found in the TME negatively impacted IL-2 therapies. Since lactic acid release by tumours is one of the main sources of acidity in the TME, we used previously described B16 melanoma cells lacking expression of lactate dehydrogenase A and B (LDHA/B) to assess the effect of pH on IL-2 therapies4. Mice were injected with B16 wild type (WT) or with B16 LDHA/B double knock-out (DKO), and treated with IL-2 conjugated to the Fc portion of human IgG4 (Fc4-IL-2) (FIG. 1c). Fc4-IL-2 therapy minimally reduced tumour growth or increased survival in mice bearing B16 WT tumours (FIG. 1d, e and Extended Data FIG. 2a). However, in mice bearing B16 DKO tumours, Fc4-IL-2 therapy significantly reduced tumour growth and increased survival (FIG. 1d, e and Extended Data FIG. 2a). Fc4-IL-2-treated mice displayed a higher percentage of infiltrating CD8+ T cells and a small increase in CD8+/Treg cell ratio as compared to the untreated mice in both B16 WT and DKO tumour (FIG. 1f, g and Extended Data FIG. 2b). No significant differences were found when Fc4-IL-2-treated B16 WT and DKO tumours were compared (FIG. 1f, g). On the other hand, CD8+ tumour-infiltrating lymphocytes (TILs) from Fc4-IL-2-treated B16 DKO tumours showed strikingly higher IFNγ and TNFα production (FIG. 1h, i and Extended Data FIG. 2c, d) and less exhausted CD8+ T cell phenotype (FIG. 1j, k) as marked by lower PD1 and TIM3 expression than Fc4-IL-2-treated B16 WT tumours. Our data show that the acid pH found in the TME profoundly inhibits IL-2 responses by blocking its binding to IL-2Rα, ultimately hindering IL-2 immunotherapy.

Engineering a pH-Resistant IL-2 Variant

Given the limitation of IL-2 to function at acidic extracellular pH, we next created mutant libraries of IL-2 conjugated to Aga2p for yeast surface display (FIG. 2a) aimed at improving binding of IL-2 to IL-2Rα at acidic pHs. In vitro directed evolution was carried out at pH 5 using decreasing concentrations of IL-2Rα ectodomain at each round (FIG. 2b) and led to the identification of a single IL-2 variant, named Switch-2, characterized by T37H, R38L, T41S, F42Y, and K43G mutations (FIG. 2c and Extended Data FIG. 3a). Switch-2 not only displayed a stronger binding to IL-2Rα at low pH but also a pH-switchable behaviour characterized by lower binding at neutral pH as compared to IL-2 WT (FIG. 2d and Extended Data FIG. 3b). Next, we assessed the ability of the IL-2 to interact with IL-2Rα on membrane of living cells at neutral (7) versus acidic pH (6) using single-molecule total internal fluorescence (TIRF) microscopy (FIG. 2e). For this purpose, IL-2Rα fused to an N-terminal SNAPf-tag tag was stably expressed in HeLa cells at physiologically relevant densities (copies/cell) and labelled by mixture of DY547 and BG-DY647 florescent dyes. Later, Dy547P1/Dy647P1 conjugated ybbR-IL-2 WT and Switch-2 was added to dynamically monitor IL-2 and IL-2Rα interaction through dual-colour co-tracking of individual cytokine receptor dimers (FIG. 2e. extended data FIG. 2c and movie S1) at single cell level. These experiments showed increased recruitment IL-2 to its IL-2Rα receptor at neutral pH, but only weak interaction at pH 6.5. By contrast, Switch-2 bore an opposing relationship, exhibiting a strong ligand-receptor interaction at acidic pH and minimal interaction at neutral pH, again confirming its pH-switchable IL-2Rα binding properties.

Next, we sought to verify if acidic pH was affecting IL-2 stability. Analysis of the thermal unfolding profiles revealed that IL-2 WT and Switch-2 exhibited comparable stabilities that were not affected by low pH (Extended Data FIG. 3d), indicating that low pH specifically inhibits IL-2 signalling by hindering cytokine-receptor interaction and not by reducing protein stability. Next, we investigated Switch-2 functionality at acidic pHs. First, we characterized the levels of STAT5 phosphorylation induced by IL-2 WT and Switch-2 in freshly isolated and pre-activated CD8+ T cells at pHs 7.5 and 6.5. In freshly isolated CD8+ T cells, which do not express IL-2Rα, both IL-2 WT and Switch-2 induced comparable STAT5 activation at pHs 6.5 and 7.5 (FIG. 2f, g). In pre-activated CD8+ T cells however, IL-2 WT triggered stronger STAT5 activation at pH 7.5 than at pH 6.5 (FIG. 2f, g). Switch-2, on the other hand, exhibited an opposite behaviour, triggering more potent STAT5 activation at pH 6.5 than at pH 7.5 (FIG. 2f, g). Similar results were obtained in kinetics studies (Extended Data FIG. 4a). Interestingly, while IL-2 WT-mediated phosphorylation of ERK1/2 was also affected by the acidic pH, in contrast, no obvious difference for L-2 mediated Akt and S6R phosphorylation were observed (Extended Data FIG. 4b-d); Switch-2 induced stronger ERK1/2 and Akt phosphorylation in pre-activated CD8+ T cells stimulated at pH 6.5 (Extended Data FIG. 4b, d). Moreover, stimulation of Treg cells with IL-2 WT and Switch-2 at pH 6.5 and 7.5 yielded comparable results as the one obtained with CD8+ T cells (Extended Data FIG. 4e).

### Switch-2 Induces Functional T Cells at Acidic pHs

IL-2 drives T cells expansion and effector functions with induction of cytotoxic function, including production of IFNγ8. However acidic pH has been reported to inhibit T cell expansion during the activation phase (Extended Data FIG. 5a)9 and T cell effector functions2. We therefore investigated the ability of CD8+ T cells stimulated with either IL-2 or Switch-2 to expand and produce effector cytokines in neutral and acidic pH conditions. CD8+ T cells were initially activated with anti-CD3 and anti-CD28 activation beads at pH 7.5, and then switch to either pH 7.5 or 6.5 media in the presence of IL-2 WT or Switch-2. As expected, while IL-2 WT induced CD8+ T cell proliferation at pH 7.5, it did not at pH 6.5 (Extended Data FIG. 5b). Switch-2, on the other hand, induced CD8+ T cell proliferation both at pH 7.5 and 6.5 (Extended Data FIG. 5b). Next, we study cytokine secretion profiles by activated CD8+ T cells stimulated with IL-2 WT or Switch-2 at pH 7.5 or 6.5. Here again, IL-2 WT elicited strong cytokine secretion by CD8+ T cells at pH 7.5, but lost almost full activity if cells were cultured at pH 6.5 (FIG. 3a). Switch-2 on the other hand, elicited almost a mirror image as compared to IL-2 WT, triggering much stronger cytokine release by CD8+ T cells at pH 6.5 than 7.5 (FIG. 3a). Moreover, cytokines associated with effector function, such as IFNγ8, GMCSF, and TNFα were secreted by cells expanded with Switch-2, while IL-2-expanded cells expressed little if any of these cytokines at the acidic pH. Next, we confirmed these results for IFNγ and TNFα using intracellular staining and flow cytometry (FIG. 3b-d).

### Switch-2 Elicits Potent Anti-Tumour Responses and Decreased Toxicity

Given its impressive effect on enhanced activity at acidic pH and reduced activity at neutral pH in a battery of in vitro assays, we hypothesize that Switch-2 may induce less systemic toxicity while inducing enhanced activity at acidic tissue niches. To test in vivo efficacy in mouse models, we first characterized Switch-2 activities in murine CD8+ T cells. Importantly, Switch-2 also triggered more potent STAT5 activation at pH 6.5 than pH 7.5 in mouse CD8+ T cells (Extended Data FIG. 6a). Considering short half-life of free IL-2, we conjugated IL-2 or “Switch-2” with a non-lytic Fc to improve functioning by extending its half-life10. To compare the effect of the IL-2 variants in mediating systemic toxicity as well as activation of immune cells in peripheral blood and tissue niches, WT C57Bl/6 mice were treated with the IL-2 variants at high-doses. One of the major side effects to high-dose IL-2 therapy is vascular leak syndrome11. As expected, high doses of IL-2 WT induced substantial pulmonary oedema (FIG. 4a). Strikingly, high doses of Switch-2 caused substantially less pulmonary oedema (FIG. 4a). This was paralleled by lower percentage of natural killer (NK) and Treg cells in the periphery in mice injected with Switch-2 (FIG. 4b and Extended Data FIG. 6 b-c), agreeing with the lower activity of Switch-2 at pH 7.5. Lymph nodes (LNs) are characterized by an acidic pH12. In agreement with this, we found that Switch-2 increased the numbers of NK and Treg cells in the LNs at higher or similar extent than IL-2 WT (FIG. 4c and Extended Data FIG. 6d). These data demonstrate Switch-2 also displayed biased activity towards acidic pH niches in vivo.

High-dose IL-2 treatment can potently activate cytotoxic T and NK cell mediated tumour killing. Yet its therapeutic efficacy is limited by poor activation of TILs within the TME. Our data demonstrate that intra-tumoral pH profoundly limited IL-2 activity within the TME (FIG. 1d-k and Extended Data FIG. 2). Indeed, large number of TILs within the tumour are dysfunctional (Res: Rosenberg SA). Importantly, TILs isolated from tumours can be reactivated and expanded in vitro the presence of IL-2. These data suggest that improper targeting and functioning of IL-2 within the TME might limit its in vivo efficacy. We hypothesize that reduced binding of Switch-2 to circulating cells at neutral pH, will improve its targeting to the activated immune cells that express CD25 within the TME and lymphoid niches. Thus, we next investigated Switch-2 anti-tumour activities in a B16 melanoma tumour model13 which in sensitive to both NK and cytotoxic T lymphocytes (CTL)-mediated killing. B16-bearing mice were injected with Fc4-IL-2 WT or Fc4-Switch-2 and tumour growth and survival rates were measured (FIG. 4d). Fc4-IL-2 WT therapy minimally reduced tumour growth and increase survival (FIG. 4e, f). Fc4-Switch-2 therapy, on the other hand, led to a strong delay in tumour growth and increase survival (FIG. 4e, f). To test differential immune response to IL-2 versus Switch-2 therapy, we analysed the B16-infiltrating T cells 4 days after the end of the therapy using flow cytometry (FIG. 4g and Extended Data FIG. 6e-g). Switch-2 and IL-2 WT triggered a small but not significant increase in the CD8-Treg ratio as compared to the PBS-treated mice (Extended Data FIG. 6e). However, Switch-2 induced a stronger CD8+ TIL proliferation and an increase in the number of infiltrating NK cells (FIG. 4g, h), a feature of effective IL-2 anti-tumour response. Moreover, Switch-2 triggered more potent IFNγ and TNFα production by CD8+ T cells than IL-2 WT (FIG. 4i, j). Notably, CD8+ TILs in Switch-2-treated groups showed substantially reduced levels of exhaustion markers as measured by expression of PD1 and TIM3 expression compared to IL-2 WT group (Extended Data FIG. 6f, g). Collectively these data show that Switch-2 induces less systemic toxicity than IL-2 WT while inducing more potent responses at the acidic TME. Importantly, these results challenge the dogma that selective binding of IL-2 to intermediate IL-2Rα affinity receptor complex is required for its anti-tumour efficacy, rather showed intra-tumoral acidic pH is a negative barrier for optimal IL-2 response through its high-affinity receptor complex that additionally expresses CD25. Furthermore, we demonstrated a rational protein engineering approach to design and develop “Switch-2” that can potently activated TILs to mediate effective immune response in poorly immunogenic B16 melanoma tumours as a single agent.

The practical implications are that IL-2 pH sensitivity can be exploited for therapy. Switch-2 robustly activates cytotoxic CD8+ T cells and NK cells for potent antitumour immune responses, yet it elicits minimal toxicity, suggesting that Switch-2 could revolutionize current IL-2 therapies.

### REFERENCES

1. Hanahan, D. & Weinberg, R. A. Hallmarks of Cancer: The Next Generation. Cell 144, 646-674 (2011).
2. Brand, A. et al. LDHA-Associated Lactic Acid Production Blunts Tumor Immunosurveillance by T and NK Cells. Cell Metabolism 24, 657-671 (2016).
3. Huber, V. et al. Cancer acidity: An ultimate frontier of tumor immune escape and a novel target of immunomodulation. Seminars in Cancer Biology 43, 74-89 (2017).
4. Renner, K. et al. Restricting Glycolysis Preserves T Cell Effector Functions and Augments Checkpoint Therapy. Cell Reports 29, 135-150.e9 (2019).
5. Mitra, S. & Leonard, W. J. Biology of IL-2 and its therapeutic modulation: Mechanisms and strategies. J. Leukoc. Biol. 103, 643-655 (2018).
6. Rosenberg, S. A. IL-2: The First Effective Immunotherapy for Human Cancer. The Journal of Immunology 192, 5451-5458 (2014).
7. Smith, K. A. & Cantrell, D. A. Interleukin 2 regulates its own receptors. PNAS 82, 864-868 (1985).
8. Pipkin, M. E. et al. Interleukin-2 and Inflammation Induce Distinct Transcriptional Programs that Promote the Differentiation of Effector Cytolytic T Cells. Immunity 32, 79-90 (2010).
9. Calcinotto, A. et al. Modulation of microenvironment acidity reverses anergy in human and murine tumor-infiltrating T lymphocytes. Cancer Res. 72, 2746-2756 (2012).
10. Mitra, S. et al. Interleukin-2 Activity Can Be Fine Tuned with Engineered Receptor Signaling Clamps. Immunity 42, 826-838 (2015).
11. Krieg, C., Létourneau, S., Pantaleo, G. & Boyman, O. Improved IL-2 immunotherapy by selective stimulation of IL-2 receptors on lymphocytes and endothelial cells. Proc Natl Acad Sci USA 107, 11906-11911 (2010).
12. Wu, H. et al. T-cells produce acidic niches in lymph nodes to suppress their own effector functions. Nature Communications 11, 4113 (2020).
13. Caudana, P. et al. IL2/Anti-IL2 Complex Combined with CTLA-4, But Not PD-1, Blockade Rescues Antitumor NK Cell Function by Regulatory T-cell Modulation. Cancer Immunol Res 7, 443-457 (2019).
14. Wang, X., Rickert, M. & Garcia, K. C. Structure of the Quaternary Complex of Interleukin-2 with Its α, β, and γc Receptors. Science 310, 1159-1163 (2005).
15. Krutzik, P. O. & Nolan, G. P. Fluorescent cell barcoding in flow cytometry allows high-throughput drug screening and signaling profiling. Nature Methods 3, 361-368 (2006).
16. Boder, E. T. & Wittrup, K. D. Yeast surface display for screening combinatorial polypeptide libraries. Nature Biotechnology 15, 553 (1997).
17. You, C., Richter, C. P., Löchte, S., Wilmes, S. & Piehler, J. Dynamic submicroscopic signaling zones revealed by pair correlation tracking and localization microscopy. Anal Chem 86, 8593-8602 (2014).
18. Moraga, I. et al. Tuning cytokine receptor signaling by re-orienting dimer geometry with surrogate ligands. Cell 160, 1196-1208 (2015).
19. Wilmes, S. et al. Receptor dimerization dynamics as a regulatory valve for plasticity of type I interferon signaling. J Cell Biol 209, 579-593 (2015).
20. Vogelsang, J. et al. A reducing and oxidizing system minimizes photobleaching and blinking of fluorescent dyes. Angew Chem Int Ed Engl 47, 5465-5469 (2008).
21. Sergé, A., Bertaux, N., Rigneault, H. & Marguet, D. Dynamic multiple-target tracing to probe spatiotemporal cartography of cell membranes. Nat Methods 5, 687-694 (2008).
22. Chen L Q, Pagel M D. Evaluating pH in the Extracellular Tumor Microenvironment Using CEST MRI and Other Imaging Methods. Adv Radiol. 2015; 2015:206405

1. A pH-resistant IL-2 mutein, wherein the amino acid sequence of the mutein comprises, relative to a wild-type IL-2 sequence, one or more amino acid modifications.

2. The IL-2 mutein of claim 1, wherein the IL-2 mutein binds IL-2Rα with higher affinity at an acidic pH.

3. The IL-2 mutein of claim 1 or 2, wherein the IL-2 mutein binds IL-2Rα with higher affinity at a pH selected from a pH of about 4.0 to about 7, preferably about 5 to about 6.5 than at a pH selected from a pH of about 7.2 to about 7.5.

4. The IL-2 mutein of claims 1 to 3, wherein the IL-2 mutein binds
(i) an IL-2 receptor or IL-2Rα at a pH of about 7.2 to about 7.5 with a lower affinity compared to a wild-type IL-2 molecule and/or
(ii) an IL-2 receptor or IL-2Rα with higher affinity at a pH selected from a pH of about 4.0 to about 7.0, preferably about 5 to about 6.5, compared to a wild-type IL-2 molecule.

5. The IL-2 mutein of claims 1-3, wherein the Il-2 mutein triggers more potent STAT5 activation at pH 6.5 than at pH 7.2.

6. The IL-2 mutein of any one of claims 1-4, wherein at an acidic pH, the mutein induces superior expansion of cytotoxic T cells as compared to a wild-type IL-2 molecule.

7. The IL-2 mutein of any one of claims 1-5, wherein at a neutral pH and as compared to a wild-type IL-2 molecule, the mutein exhibits reduced IL-2Rα binding and/or a reduced ability to expand cytotoxic T cells and/or less potent STAT5 activation.

8. The IL-2 mutein of any preceding claim, wherein the wild-type IL-2 sequence comprises SEQ ID NO: 1, 3 to 8.

9. The IL-2 mutein of any preceding claim, wherein the mutein comprises an amino acid substitution at residue 37 of SEQ ID NO: 1, 4, 5, 8 or respective residues in SEQ ID NO: 3, 6, or 7.

10. The IL-2 mutein of claim 8, wherein the mutein comprises a non-conservative or conservative substitution, preferably the non-conservative substitution is to a positively charged amino acid at residue 37 of SEQ ID NO: 1, 4, 5, 8 or respective residues in SEQ ID NO: 3, 6, or 7.

11. The IL-2 mutein of claim 10, wherein the mutein comprises a threonine to histidine, arginine or serine substitution at residue 37 of SEQ ID NO: 1, or 8.

12. The IL-2 mutein of any one of claims 1-8, wherein the mutein comprises an amino acid substitution at residue 38 of SEQ ID NO: 1, 4, 5, 8 or respective residues in SEQ ID NO: 3, 6, or 7.

13. The IL-2 mutein of claim 12, wherein the mutein comprises a non-conservative or conservative substitution, preferably an arginine to leucine, valine, isoleucine or alanine substitution at residue 38 of SEQ ID NO: 1, 4, 5, 8 or respective residues in SEQ ID NO: 3, 6, or 7.

14. The IL-2 mutein of any one of claims 1-13, wherein the mutein comprises an amino acid substitution at residue 41 of SEQ ID NO: 1, 4, 5, 8 or respective residues in SEQ ID NO: 3, 6, or 7.

15. The IL-2 mutein of claim 14, wherein the mutein comprises a conservative or non-conservative substation, preferably a threonine to serine, glycine or aspartic acid substitution at residue 41 of SEQ ID NO: 1, 4, 5, 8 or respective residues in SEQ ID NO: 3, 6, or 7.

16. The IL-2 mutein of any one of claims 1-15 wherein the mutein comprises an amino acid substitution at residue 42 of SEQ ID NO: 1, 4, 5, 8 or respective residues in SEQ ID NO: 3, 6, or 7.

17. The IL-2 mutein of claim 16, wherein the mutein comprises a non-conservative substation, preferably a substitution to a polar neutral amino acid at residue 42 of SEQ ID NO: 1, 4, 5, 8 or respective residues in SEQ ID NO: 3, 6, or 7.

18. The IL-2 mutein of claim 17, wherein the mutein comprises a phenylalanine to tyrosine substitution at residue 42.

19. The IL-2 mutein of any one of claims 1-18, wherein the mutein comprises an amino acid substitution at residue 43 of SEQ ID NO: 1, 4, 5, 8 or respective residues in SEQ ID NO: 3, 6, or 7.

20. The IL-2 mutein of claim 19, wherein the mutein comprises a non-conservative substitution, preferably a substitution against a polar neutral amino acid at residue 43 of SEQ ID NO: 1, 4, 5, 8 or respective residues in SEQ ID NO: 3, 6, or 7.

21. The IL-2 mutein of claim 20, wherein the mutein comprises a lysine to glycine substitution at residue 43 of SEQ ID NO: 1, 4, 5, 8 or respective residues in SEQ ID NO: 3, 6, or 7.

22. The IL-2 mutein of any one of claims 1-21, wherein the mutein comprises an amino acid substitution at residue 64 of SEQ ID NO: 1, 4, 5, 8 or respective residues in SEQ ID NO: 3, 6, or 7.

23. The IL-2 mutein of any one of claims 1-22, wherein the mutein comprises a conservative or non-conservative amino acid substitution, preferably a substitution against an acidic amino acid at residue 64 of SEQ ID NO: 1, 4, 5, 8 or respective residues in SEQ ID NO: 3, 6, or 7.

24. The IL-2 mutein of claim 23, wherein the mutein comprises a lysine to glutamic acid substitution at residue 64.

25. The IL-2 mutein of any one of claims 9-24, wherein the mutein further comprises one or more modifications at one or more other residues.

26. The IL-2 mutein of claim 25, wherein the mutein further comprises one or more modifications at any of positions 37, 38, 41, 42, 43, and/or 65 of SEQ ID NO: 1, 4, 5, 8 or respective residues in SEQ ID NO: 3, 6, or 7.

27. The IL-2 mutein of any one of claims 9-24, wherein the mutein comprises at least a modification at positions 37, 38, 41, 43 and at least one further modification selected from positions 42 and 64 of SEQ ID NO: 1, 4, 5, 8 or respective residues in SEQ ID NO: 3, 6, or 7.

28. The IL-2 mutein of any one of claims 1-8, wherein relative to the sequence of SEQ ID NO: 1, the IL-2 mutein comprises amino acid modifications to the each of the residues at positions 37, 38, 41, 42 and 43 of SEQ ID NO: 1, 4, 5, 8 or respective residues in SEQ ID NO: 3, 6, or 7.

29. The IL-2 mutein of any one of claims 1 to 8, wherein the IL-2 mutein comprises amino acid modifications to the each of the residues at positions 37, 38, 41, 43 and 64 of SEQ ID NO: 1, 4, 5, 8 or respective residues in SEQ ID NO: 3, 6, or 7.

30. The IL-2 mutein of any one of claims 1-8, wherein relative to the sequence of SEQ ID NO: 1 or 8, the IL-2 mutein comprises one, two, three, four or all five of the following amino acid substitutions:
(i) T37H; and/or
(ii) R38L; and/or
(iii) T41S; and/or
(iv) F42Y; and/or
(v) K43G.

31. The IL-2 mutein of any one of claims 1-8, wherein relative to the sequence of SEQ ID NO: 1 or 8, the IL-2 mutein comprises one, two, three, four or all five of the following amino acid substitutions:
(i) T37S; and/or
(ii) R38A; and/or
(iii) T41D; and/or
(iv) K43G; and/or
(v) K65E.

32. The IL-2 mutein of any one of claims 1-8, wherein relative to the sequence of SEQ ID NO: 1 or 8, the IL-2 mutein comprises one, two, three, four or all five of the following amino acid substitutions:
(i) T37S; and/or
(ii) R38L; and/or
(iii) T41G; and/or
(iv) F42Y; and/or
(v) K43G.

33. The IL-2 mutein of any one of claims 1-8, wherein relative to the sequence of SEQ ID NO: 1 or 8, the IL-2 mutein comprises one, two, three, four or all five of the following amino acid substitutions:
(i) T37S; and/or
(ii) R38V; and/or
(iii) T41G; and/or
(iv) K43G.

34. The IL-2 mutein of any one of claims 1-8, wherein relative to the sequence of SEQ ID NO: 1 or 8, the IL-2 mutein comprises one, two, three, four or all five of the following amino acid substitutions:
(i) T37R; and/or
(ii) R38V; and/or
(iii) T41G; and/or
(iv) K43G.

35. The IL-2 mutein of any one of claims 1-8, wherein relative to the sequence of SEQ ID NO: 1 or 8, the IL-2 mutein comprises one, two, three, four or all five of the following amino acid substitutions:
(i) T37S; and/or
(ii) R38I; and/or
(iii) T41G; and/or
(iv) K43G.

36. The IL-2 mutein of any one of claims 1-8, wherein the mutein comprises SEQ ID NO: 2, 9, 10, 11, 12 or 13 or a functional fragment thereof.

37. A fusion protein comprising an IL-2 mutein according to any preceding claim or a functional fragment thereof.

38. The fusion protein of claim 37, wherein the fusion protein comprises a further polypeptide molecule or polypeptide fragment.

39. The fusion protein of claim 38, wherein the further polypeptide molecule or peptide fragment is fused to the IL-2 mutein or the functional fragment thereof.

40. The fusion protein according to any one of claims 38-39, wherein the further polypeptide molecule or polypeptide fragment:
a cytokine or fragment thereof; or
an interleukin molecule or fragment thereof.

41. The fusion protein according to claims 37-39, wherein the further polypeptide molecule or polypeptide fragment comprises:
a polypeptide binding domain;
an antibody or a fragment thereof;
a single chain antibody; or
a VHH.

42. The fusion protein according to claim 41, wherein the polypeptide binding domains binds to a tumor antigen or a checkpoint molecule.

43. The fusion protein according to claims 41-42, wherein the polypeptide binding domains binds to at least one checkpoint molecule selected from CD27, CD137, 2B4, TIGIT, CD155, CD160, ICOS, HVEM, CD40L, LIGHT, LAIR1, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDO1, ID02, TDO, KIR, LAG-3, TIM-3, or VISTA.

44. The fusion protein according to claim 41, wherein the polypeptide binding domains binds to an antigen expressed by a regulatory T-cell.

45. The fusion protein according to claim 41, wherein the antibody: is an antagonistic antibody or antagonistic fragment thereof; or
an agonistic antibody or an agonistic fragment thereof.

46. The fusion protein according to claim 41 or 45, wherein the antibody or fragment thereof is a pH sensitive antibody or fragment.

47. The fusion protein according to any one of claims 37-46, wherein the IL-2 mutein is fused to a polypeptide domain that renders the IL-2 mutein conditionally inactive.

48. The fusion protein according to claim 37-46, wherein the IL-2 mutein or functional fragment is fused to a half-life extending molecule, optionally wherein the half-life extending molecule comprises an immunoglobulin fragment, an Fc molecule, a polypeptide binding domain which binds a blood serum protein, a polypeptide binding domain which binds albumin, or a polymer.

49. The fusion protein of claim 48, wherein the polymer comprises a polyethylene glycol molecule.

50. A nucleic acid encoding an IL-2 mutein or fusion protein according to any one of claims 1-49.

51. A nucleic acid encoding SEQ ID NO: 2, 9, 10, 11, 12 or 13.

52. A vector comprising a nucleic acid according to claim 50 or claim 51.

53. A host cell transformed with the nucleic acid of claims 50-51 or vector of claim 52.

54. The host cell of claim 53, wherein the host cell is a T-cell, preferably a T cell comprising a chimeric antigen receptor (CAR).

55. A method of making an IL-2 mutein, said method comprising introducing a nucleic acid or vector according to any one of claims 50-52 into a host cell and expressing the nucleic acid.

56. A method of identifying a pH-resistant cytokine mutein, said method comprising:
mutating or modifying cytokine molecule to generate a cytokine mutein; and
contacting the cytokine mutein with a cytokine receptor under acidic conditions so as to identify mutein(s) which bind to the cytokine receptor and are pH resistant.

57. The method of claim 56, wherein the pH resistant cytokine mutein is an IL-2 mutein.

58. A pH-resistant cytokine mutein obtainable by a method comprising mutating or modifying a wild-type cytokine molecule to generate a cytokine mutein; and contacting the cytokine mutein with a ligand for that cytokine under acidic conditions, wherein any cytokine mutein which is found to bind to the ligand under acidic conditions is a pH resistant cytokine mutein.

59. A pH-resistant IL-2 mutein obtainable by a method comprising mutating or modifying a wild-type IL-2 molecule to generate an IL-2 mutein; and contacting the IL-2 mutein with IL-2Rα under acidic conditions, wherein any IL-2 mutein which is found to bind to IL-2Rα under acidic conditions is a pH resistant IL-2 mutein.

60. An IL-2 mutein, fusion protein or cytokine mutein according to any one of claims 1-49, 58 or 59, for use in medicine.

61. A protein, fusion protein or composition comprising SEQ ID NO: 2, 9, 10, 11, 12 or 13 or a fragment thereof, for use in medicine.

62. A nucleic acid according to anyone of claims 50-51, for use in medicine.

63. A nucleic acid encoding a protein comprising SEQ ID NO: 2, 9, 10, 11, 12 or 13 or a fragment thereof, for use in medicine.

64. An IL-2 mutein, fusion protein, nucleic acid or cytokine mutein according to anyone of claims 1-51, 58 or 59 for use in the treatment or prevention of an immunological condition.

65. An IL-2 mutein, fusion protein, nucleic acid or cytokine mutein according to anyone of claims 1-51, 58 or 59 for use in the treatment or prevention of cancer.

66. An IL-2 mutein, fusion protein, nucleic acid or cytokine mutein according to anyone of claims 1-51, 58 or 59 for use in the treatment or prevention of infectious diseases.

67. An IL-2 mutein, fusion protein, nucleic acid or cytokine mutein according to anyone of claims 1-51, 58 or 59 for use as an adjuvant, optionally wherein the adjuvant is a vaccine adjuvant.

68. A composition comprising an IL-2 mutein, a fusion protein, nucleic acid or a cytokine mutein according to anyone of claims 1-51, 58 or 59.

69. The composition of claim 68, wherein the composition is a pharmaceutical composition, optionally including one or more pharmaceutically acceptable excipients.

70. The composition of any one of claims 68 and 69, wherein the composition or pharmaceutical composition comprises another therapeutic moiety or pharmaceutically active agent.

71. A method of treating or preventing an immunological condition and/or cancer, said method, comprising administering a subject in need thereof a therapeutically effective amount of an IL-2 mutein, fusion protein, cytokine mutein, or nucleic acid according to anyone of claims 1-51, 58 or 59.

72. The method of treating or preventing an immunological condition and/or cancer according to claim 71, wherein the IL-2 mutein, fusion protein, cytokine mutein, or nucleic acid is administered in combination with an anti-tumour antigen antibody, a checkpoint molecule, an antibody against a checkpoint molecule, a tumour antigen a steroid and/a CAR T-cell.

73. The method of claim 72, wherein the checkpoint molecule or antibody is directed against a checkpoint molecule selected from CD27, CD137, 2B4, TIGIT, CD155, CD160, ICOS, HVEM, CD40L, LIGHT, LAIR1, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDO1, ID02, TDO, KIR, LAG-3, TIM-3, or VISTA.

74. The method of treating or preventing an immunological condition and/or cancer according to claims 71 to 73, comprising a step of determining the extracellular pH of the TME in a patient prior to administering the IL-2 mutein, fusion protein, cytokine mutein, or nucleotide.

75. The host cell of claim 54, for use in treating or preventing an immunological condition and/or cancer.

76. Use of the host cell of claim 54, in the manufacture of a medicament for treating or preventing an immunological condition and/or cancer.

77. A method of treating or preventing an immunological condition and/or cancer, said method comprising administering a subject in need thereof a host cell according to claim 54.
